(19) United States
(12) Patent Application Publication
(10) Pub. No.: US 2016/0024536 A1
(43) Pub. Date: Jan. 28, 2016

(54) GENETICALLY ENGINEERED AND ACID-RESISTANT YEAST CELL WITH ENHANCED ERG5 ACTIVITY AND METHOD OF PRODUCING LACTATE BY USING THE YEAST CELL

(71) Applicant: Samsung Electronics Co., Ltd., Suwon-si (KR)

(72) Inventors: Wooyong Lee, Hwaseong-si (KR); Soyoung Lee, Daejeon (KR); Huisub Lim, Seoul (KR); Jiyoon Song, Seoul (KR); Kwangmyung Cho, Seongnam-si (KR); Sunghaeng LEE, Seoul (KR)

(21) Appl. No.: 14/657,637

(22) Filed: Mar. 13, 2015

(30) Foreign Application Priority Data
Jul. 24, 2014 (KR) 10-2014-0094158

(51) Int. Cl. C12P 7/56; C12N 15/81

(52) U.S. Cl. CPC C12P 7/56; C12N 15/81

(57) ABSTRACT

Provided is a recombinant acid resistance yeast cell that is genetically engineered to increase ERG5 activity and a method of producing lactate by using the yeast cell.

### RELATED APPLICATION

This application claims the benefit of Korean Patent Application No. 10-2014-0094158, filed on Jul. 24, 2014, in the Korean Intellectual Property Office, the entire disclosure of which is hereby incorporated by reference.

### INCORPORATION-BY-REFERENCE OF MATERIAL ELECTRONICALLY SUBMITTED

Incorporated by reference in its entirety herein is a computer-readable nucleotide/amino acid sequence listing submitted herewith and identified as follows: One 88,212 Bytes ASCII (Text) file named “719229_ST25.TXT,” created on Mar. 2, 2015.

### BACKGROUND

1. Field

The present disclosure relates to a genetically engineered (i.e., recombinant) and acid-resistant yeast cell with enhanced ERG5 activity and a method of producing lactate by using the yeast cell.

2. Description of the Related Art

Organic acids are widely used in a variety of industries. For example, lactate is an organic acid that is used in a variety of industrial fields, including the food, pharmaceutical, chemical, and electronic industries. Lactate is a colorless, odorless, water-soluble, low-volatile material. Lactate is also not toxic to the human body, and is used as a flavoring agent, a sour taste agent, a preserving agent, or the like. Lactate is also used as a source of polylactic acid (PLA) that is an environmentally friendly, biodegradable plastic known as an alternate polymeric material.

Organic acids may be dissociated into hydrogen ions and their own negative ions at a pH higher than their own dissociation constant (pka value), for example, under a neutral condition. Meanwhile, organic acids, for example, lactic acid, may be present in the form of free acid without an electromagnetic force at a pH lower than its own pKa value. An organic acid in the form of negative ions may not be permeable with respect to a cell membrane, but may be permeable with respect to the cell membrane when it is present in the form of free acid. Therefore, an organic acid in free acid form may flow into the cells from extracellular environments where the concentration of the organic acid is high, and thus lower an intercellular pH level. Meanwhile, an organic acid present as negative ions requires an additional isolation process involving the addition of a salt. Also, a cell lacking acid-resistance may become inactive and die under acidic conditions, such as in the case of lactic acid.

Therefore, there is a need for a microorganism with acid-resistance.

### SUMMARY

Provided is an acid-resistant recombinant yeast cell that is genetically engineered to have enhanced ERG5 activity as compared to a parent yeast cell.

Provided is a method of producing lactate by culturing the recombinant yeast cell such that the recombinant yeast cell produces lactate in culture.

Further provided is a method of preparing an acid-resistant recombinant yeast cell comprising introducing into a yeast cell an exogenous nucleic acid encoding ERG5, or increasing the copy number of an endogenous nucleic acid encoding ERG5 in the yeast cell.

Additional aspects will be set forth in part in the description which follows and, in part, will be apparent from the description, or may be learned by practice of the presented embodiments.

### BRIEF DESCRIPTION OF THE DRAWINGS

These and/or other aspects will become apparent and more readily appreciated from the following description of the embodiments, taken in conjunction with the accompanying drawings in which:

FIG. 1A is a vector map of a pCS-Ex1 vector.

FIG. 1B is a vector map of a pCS-EX1 ERG5 vector;

FIG. 2 shows the results of culturing various yeast cells on a pH controlled-YPD acid medium including lactic acid (“LA”); and

FIG. 3 is a graph illustrating concentrations of lactate and glucose of an ERG5 gene overexpressed yeast cell (circle) and its control group (square). In FIG. 3, the filled symbols represent L-lactic acid concentrations, and the unfilled symbols represent D-Glucose concentrations.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

As used herein, the terms “activity increase” or “increased activity” and the like in reference to a cell, an enzyme, a polypeptide, or a protein refers to an increased amount of an enzyme, a polypeptide, or a protein sufficient to increase the expressed (e.g., detectable) activity thereof, and denotes a cell or a polypeptide whose activity is increased compared to a cell or polypeptide of the same type, such as the parent cell of a recombinant cell, or original polypeptide. For instance, an increase in activity of a recombinant or genetically engineered polypeptide or cell may be about 5% or more, about 10% or more, about 15% or more, about 20% or more, about 30% or more, about 50% or more, about 60% or more, about 70% or more, about 100%, about 200%, or about 300% or more compared to the activity of a non-recombinant or non-engineered polypeptide or cell, such as a parent cell of the recombinant cell or a polypeptide of the parent cell, e.g., a wild-type polypeptide or cell. Enhanced activity of a polypeptide or cell may be confirmed by using any method commonly known in the art, for example the activity of ERG5 enzyme may be measured by using isotope.

Increased activity of the polypeptide may result from expression increase or increased specific activity of the polypeptide. The expression increase may be may be caused by introduction of an exogenous polynucleotide encoding a polypeptide into a cell, by increasing the copy number of an endogenous polynucleotide in the cell, or by mutation of a regulatory region of the polynucleotide. The mutation of a regulatory region of the polynucleotide may include modification of an expression regulatory sequence of a gene. The regulatory sequence may be a promoter sequence for expression of the gene or a transcription terminator sequence. Also, the regulatory sequence may be a sequence encoding a motif that may affect the gene expression. Examples of the motif may include 2D stabilizing motifs, RNA instability motifs, splice-activating motifs, polyadenylation motifs, adenine-rich sequences, and endonuclease recognition sites.

An endogenous gene may refer to a gene present in a genetic material contained within the microorganism prior to genetic manipulation. An exogenous gene may refer to a gene that is introduced to a host cell from outside the cell. The exogenous gene, once introduced, may integrate into a host cell genome. An exogenous gene may contain a genetic sequence that is homologous or heterologous with respect to the host cell. The term “homologous” means that the gene is of the genetic origin as the host cell, whereas “heterologuos” denotes a gene that is foreign to the host cell (derived from a different genetic origin and, thus, non-native to the host cell).

The expression “increased copy number,” “copy number increase,” and the like may include a case where a copy number increase is achieved by an introduction or amplification of the gene and a case where a copy number increase by genetically engineering a cell that does not exist in a genetically non-engineered (i.e., parent) cell. The introduction of the gene may occur by using a vehicle such as a vector. The introduction may be a transient introduction, in which the gene is not integrated into the genome, or integration into the genome. The introduction may, for example, occur by introducing a vector containing a polynucleotide encoding a desired polypeptide into the cell, and then replicating the vector in the cell, or integrating the polynucleotide into the genome of the cell and then replicating the polynucleotide together with the replication of the genome.

As used herein, the term “gene” denotes a nucleic acid that encodes a gene product (e.g., mRNA or protein) when expressed (e.g., transcribed and translated). A “gene” as used herein encompasses, but is not limited to, a genomic sequence as well as any other nucleic acid encoding a given gene product. The term “gene,” as defined herein, does not imply or require the presence of regulatory elements, although a gene may comprise regulatory elements. An example of a gene product is mRNA or other nucleic acid that can be translated to produce a protein, which nucleic acid may include a coding region or a regulatory sequence of a 5′-non coding sequence and a 3′-non coding sequence in addition to the coding region.

The terms “cell”, “strain”, or “microorganism” as used herein may be interchangeably used and may include bacteria, yeast, or fungi.

As used herein, the terms “activity reduction”, “reduced activity”, “decreased activity” and the like, in reference to a cell, an enzyme, a protein, or a polypeptide denotes a cell, an enzyme, a protein, or a polypeptide whose activity is measurably lower than the activity measured in a comparable cell, enzyme, protein, or polypeptide of the same type. Reduced activity includes a cell, an isolated enzyme, or a polypeptide having no activity. For example, the activity of a recombinant or genetically engineered polypeptide or cell may be reduced by about 10% or more, about 20% or more, about 30% or more, about 40% or more, about 50% or more, about 55% or more, about 60% or more, about 70% or more, about 75% or more, about 80% or more, about 85% or more, about 90% or more, about 95% or more, or about 100% as compared to the activity of a polypeptide or cell that is not genetically engineered, such as a parent cell of the genetically engineered cell or a polypeptide of the parent cell (e.g., a wild-type polypeptide or cell). The decreased activity may be confirmed by using any commonly known method in the art. The decrease in activity may include the case of an enzyme having no activity or reduced activity even when the enzyme is expressed, a gene encoding the enzyme that is not expressed, or a decrease in an expression amount of the gene compared to that of a gene encoding an originally not engineered polypeptide or a wild-type polypeptide even when the gene encoding the enzyme is expressed.

Activity of the enzyme may be reduced due to a deletion or disruption mutation of a gene that encodes the enzyme. As used herein, the “deletion” or “disruption” of the gene includes the case where all or part of a gene, or all or part of a regulatory region (e.g., a promoter or terminator of the gene) is mutated (substitution, deletion, or insertion) to reduce or eliminate expression, to reduce or eliminate activity of the enzyme even when the gene is expressed. The deletion or disruption of the gene may be accomplished by genetic engineering techniques, such as homologous recombination, mutation induction, or molecular evolution. When a cell includes a plurality of the same genes, or at least two different polypeptide paralogs, at least one gene may be deleted or disrupted.

As used herein, the term “sequence identity” of a polypeptide or polynucleotide with respect to another polypeptide or polynucleotide refers to a degree of sameness in the amino acid residues or a nucleotide bases in a specific region of two sequences that are aligned to best match each other for comparison. The sequence identity is a value obtained via optimal alignment and comparison of the two sequences in the specific region for comparison, in which a partial sequence in the specific region for comparison may be added or deleted with respect to a reference sequence. The sequence identity represented in a percentage may be calculated by, for example, comparing two sequences that are aligned to best match each other in the specific region for comparison, determining matched sites with the same amino acid or base in the two sequences to obtain the number of the matched sites, dividing the number of the matched sites in the two sequences by a total number of sites in the compared specific regions (i.e., a size of the compared region), and multiplying a result of the division by 100 to obtain a sequence identity as a percentage. The sequence identity as a percentage may be determined using a known sequence comparison program, for example, BLASTN (NCBI), CLC Main Workbench (CLC bio), or MegAlign™ (DNASTAR Inc).

In identifying a polypeptide or polynucleotide with the same or similar function or activity with respect to various types of species, any various levels of sequence identity may be applied. In some embodiments, the sequence identity may be, for example, about 50% or more, 55% or more, 60% or more, 65% or more, 70% or more, 75% or more, 80% or more, 85% or more, 90% or more, 95% or more, 96% or more, 97% or more, 98% or more, 99% or more, or 100%.

As used herein, the term “parent cell” may denote a cell without a given specific genetic modification, e.g., the genetic modification of the ERG5, that provides a particular genetically engineered cell. Also, the term “wild-type” polypeptide or polynucleotide may denote a polypeptide or a polynucleotide without a specific genetic modification that provides a genetically engineered polypeptide or polynucleotide. For example, the parent cell of a cell that is genetically engineered to increase ERG5 activity can be a cell that is lacking the genetic modification that increases activity of ERG5 in the genetically engineered cell. The parent cell may be a strain that is genetically modified to increase activity of ERG5. In other words, the parent cell can be the starting material from which a genetically engineered strain is made. Thus, the parent cell may be a cell without a genetic modification that increases activity of ERG5.

As used herein, the term “lactate” is interpreted as including its anion form, a salt thereof, a solvate, a polymorph, or a combination thereof in addition to lactic acid itself. The salt may be, for example, an inorganic acid salt, an organic acid salt, or a metal salt. The inorganic acid salt may be a hydrochloride, bromate, phosphate, sulfate, or disulfate. The inorganic acid salt may be formate, acetate, propionate, lactate, oxalate, tartrate, malate, maleate, citrate, fumarate, besylate, camsylate, edisylate, trifluoroacetate, benzoate, gluconate, methansulfonate, glycolate, succinate, 4-toluenesulfonate, galacturonate, embonate, glutamate, or aspartate. The metal salt may be a calcium salt, a sodium salt, a magnesium salt, a strontium salt, or a potassium salt.

According to an exemplary embodiment, a recombinant acid-resistant yeast cell has increased ERG5 activity compared to that of a parent cell.

ERG5 may be a C-22 sterol desaturase or Cytochrome P450 61 (EC: 1.14.-.-). The C-22 sterol desaturase may catalyze formation of a catalyst forming a C-22(23) double bond in a sterol side chain. ERG5 may include an amino acid sequence having about 60% or more, 70% or more, 80% or more, 90% or more, 95% or more, 96% or more, 97% or more, 98% or more, or 99% or more sequence identity with an amino acid sequence of SEQ ID NO: 1. The ERG5 may be encoded by a polynucleotide that encodes a protein that has at least 95% sequence identity with SEQ ID NO: 1, or may be a polynucleotide having 95% or more sequence identity with SEQ ID NO: 2.

The recombinant yeast cell with an acid-resistant property of may have a better growth rate under an acid condition compared to the growth of a parent cell. The acid condition may be an acid condition including an organic acid, an inorganic acid, or a combination thereof. The organic acid may be an organic acid having 1 to 20 carbon atoms. The organic acid may be acetic acid, lactic acid, propionic acid, 3-hydroxypropionic acid, butyric acid, 4-hydroxybutyric acid, succinic acid, fumaric acid, malic acid, oxalic acid, adipic acid, or a combination thereof. The yeast cell may be better growing under a pH condition in a range of about 2.0 to about 7.0, for example, pH in a range of about 2.0 to about 5.0, about 2.0 to about 4.5, about 2.0 to about 4.0, about 2.0 to about 3.8, about 2.5 to about 3.8, about 3.0 to about 3.8, about 2.0 to about 3.0, about 2.0 to about 2.7, about 2.0 to about 2.5, or about 2.5 to about 3.0 compared to that of a yeast cell in which ERG5 activity is not increased. The degree of growth may be measured by counting of microorganism colonies or measuring the optical density (OD) of the colonies. The yeast cell may have increased growth rate as measured by OD compared to that of a yeast cell in which ERG5 activity is not increased.

Also, the recombinant yeast cell with an acid-resistant property of may have a higher survival rate under an acid condition compared to that of a parent cell. The acid condition may be an acid condition including an organic acid, an inorganic acid, or a combination thereof. The organic acid may be an organic acid having 1 to 20 carbon atoms. The organic acid may be acetic acid, lactic acid, propionic acid, 3-hydroxypropionic acid, butyric acid, 4-hydroxybutyric acid, succinic acid, fumaric acid, malic acid, oxalic acid, adipic acid, or a combination thereof. The recombinant yeast cell may have a higher survival rate under a pH condition in a range of about 2.0 to about 7.0, for example, pH in a range of about 2.0 to about 5.0, about 2.0 to about 4.5, about 2.0 to about 4.0, about 2.0 to about 3.8, about 2.5 to about 3.8, about 3.0 to about 3.8, about 2.0 to about 3.0, about 2.0 to about 2.7, about 2.0 to about 2.5, or about 2.5 to about 3.0 compared to that of a yeast cell in which ERG5 activity is not increased.

Also, the recombinant yeast cell with an acid-resistant property may have higher metabolization ability at an acid condition compared to that of a parent cell. The metabolization may mean chemical transformations (e.g., enzyme-catalyzed reactions) within the yeast cell. The enzyme-catalyzed reactions may allow the yeast cell to grow, reproduce, and respond to their environment such as acidic condition. The metabolization ability may be determined by measuring a consumption of one or more nutrients such as hexose (e.g., glucose) or pentose, or production of one or more metabolites such as lactate in the cell. The acid condition may be an acid condition including an organic acid, an inorganic acid, or a combination thereof. The organic acid may be an organic acid having 1 to 20 carbon atoms. The organic acid may be acetic acid, lactic acid, propionic acid, 3-hydroxypropionic acid, butyric acid, 4-hydroxybutyric acid, succinic acid, fumaric acid, malic acid, oxalic acid, adipic acid, or a combination thereof. The recombinant yeast cell may have higher metabolization ability under a pH condition in a range of about 2.0 to about 7.0, for example, pH in a range of about 2.0 to about 5.0, about 2.0 to about 4.5, about 2.0 to about 4.0, about 2.0 to about 3.8, about 2.5 to about 3.8, about 3.0 to about 3.8, about 2.0 to about 3.0, about 2.0 to about 2.7, about 2.0 to about 2.5, or about 2.5 to about 3.0 compared to that of a yeast cell in which ERG5 activity is not increased. Here, a degree of “metabolization ability” may be measured by a nutrition uptake rate per cell, for example, a glucose uptake rate per cell. Also, a degree of “metabolization ability” may be measured by a product secretion rate per cell, for example, a carbon dioxide secretion rate per cell.

The term “acid-resistant”, “acid-tolerant”, “acid tolerating”, “acid-resistance”, and “acid tolerance” may be used interchangeably.

The recombinant yeast cell may have modification in an expression regulatory sequence of a gene encoding ERG5. The expression regulatory sequence of the gene may be a promoter sequence for expression of the gene or a transcription terminator sequence. Also, the expression regulatory sequence may be a sequence encoding a motif that may affect the gene expression. Examples of the motif may include 2D stabilizing motifs, RNA instability motifs, splice-activating motifs, polyadenylation motifs, adenine-rich sequences, and endonuclease recognition sites.

The promoter sequences may be exogenous promoter that is operably linked to the gene encoding ERG5. The promoter may be a constitutive promoter. The promoter may be a promoter derived from a Covalently linked Cell Wall protein 12 (CCW12), Pyruvate DeCarboxylase 1(PDC1), phosphoglycerate kinase (PGK1), Transcription enhancer factor-1(TEF-1), glyceraldehyde-3-phosphate dehydrogenase (TDH1, TDH2, or TDH3), triose phosphate isomerase (TPI1), purine-cytosine permease (PCPL3), or alcohol dehydrogenase (ADH1) gene. Also, the expression regulatory sequence may be a sequence that improves translation efficiency. The sequence improving translation efficiency may be, for example, a Kozak consensus sequence, which improves initiation of a translation process. The Kozak consensus sequence may be, for example, AAACA. In one embodiment, the Kozak consensus sequence may be inserted in a promoter. The promoter may be selected from the group consisting of a cytochrome c (CYC) promoter, a transcription elongation factor (TEF) promoter, a glycerol-3-phosphate dehydrogenase (GPD) promoter, an alcohol dehydrogenase (ADH) promoter, and a promoter of CCW12 gene. In another embodiment, the Kozak consensus sequence may be inserted in a regulatory region (for example, promoter) of ERG5 gene.

Also, the yeast cell may have an increased copy number of the gene encoding ERG5. The recombinant yeast cell may include an exogenous gene that encodes ERG5. The exogenous gene may be appropriately controlled by an exogenous promoter that is operably linked with a gene. The promoter is the same as described above.

The recombinant yeast cell may belong to Saccharomyces genus, Kluyveromyces genus, Candida genus, Pichia genus, Issatchenkia genus, Debaryomyces genus, Zygosaccharomyces genus, Shizosaccharomyces genus, or Saccharomycopsis genus. Saccharomyces genus may be, for example, S. cerevisiae, S. bayanus, S. boulardii, S. bulderi, S. cariocanus, S. cariocus, S. chevalieri, S. dairenensis, S. ellipsoideus, S. eubayanus, S. exiguus, S. florentinus, S. kluyveri, S. martiniae, S. monacensis, S. norbensis, S. paradoxus, S. pastorianus, S. spencerorum, S. turicensis, S. unisporus, S. uvarum, or S. zonatus.

The increase in activity of ERG5 may be due to the increased copy number of the gene encoding one or more genes or due to modification of the expression regulatory sequence of the gene. The regulatory sequence in gene expression may include a sequence of a promoter for the gene expression or a sequence of a transcription terminator. The sequence of the promoter may be an exogenous promoter that is operably linked to a gene encoding the EDC protein. The promoter may be modified to a constitutive promoter. The constitutive promoter may be derived from GPD, covalently linked cell wall protein 12 (CCW12), pyruvate deCarboxylase 1 (PDC1), phosphoglycerate kinase (PGK1), transcription enhancer factor-1 (TEF-1), glyceraldehyde-3-phosphate dehydrogenase (TDH1, TDH2, or TDH3), triose phosphate isomerase (TPI1), purine-cytosine permease (PCPL3), or alcohol dehydrogenase (ADH1) genes. In addition, the regulatory sequence in gene expression may be modified to include a sequence that improves efficiency of translation. The sequence that improves efficiency of translation may be, for example, a sequence that improves initiation of the translation process, such as Kozak consensus sequence. The increased copy number may be caused by introduction of the gene from outside to inside of the cell or by amplification of the endogenous gene.

The introduction of an exogenous gene may be performed by mediation of vehicles, such as a vector. The introduction may be transient introduction that is not integrated to a genome or by insertion into a genome. For example, the introduction may be performed by introducing a vector, to which the gene is inserted, to a cell, and copying the vector in the cell or integrating the gene into the genome. The gene may be operably linked to a regulatory sequence involved in regulation of expression of the gene. The regulatory sequence may include a promoter, a 5′-non coding sequence, a 3′-non coding sequence, an transcription terminator sequence, an enhance, or a combination thereof. The gene may be an endogenous gene or an exogenous gene. Also, the regulatory sequence may be a sequence encoding a motif that may affect the gene expression. Examples of the motif may include 2D stabilizing motifs, RNA instability motifs, splice-activating motifs, polyadenylation motifs, adenine-rich sequences, and endonuclease recognition sites.

The increase in activity of ERG5 may be caused by mutation of an endogenous gene encoding one or more enzymes. The mutation may cause substitution, insertion, addition, or conversion of at least one base.

The recombinant yeast cell may be capable of producing lactate. The recombinant yeast cell may have an activity of a polypeptide that converts pyruvate into lactate. The recombinant yeast cell may include a gene encoding a polypeptide that converts pyruvate into lactate. In the recombinant yeast cell, the activity of a polypeptide that converts pyruvate into lactate may be increased as compared to a parent cell. The polypeptide that converts pyruvate into lactate may be a lactate dehydrogenase (LDH). The LDH may be an NAD(P)-dependent enzyme. Also, the LDH may be stereo-specific and may produce only L-lactate, only D-lactate, or both L-lactate and D-lactate. The NAD(P)-dependent enzyme may be an enzyme that is classified under EC 1.1.1.27 that functions on L-lactate or EC 1.1.1.28 that functions on D-lactate.

The yeast cell may include a gene encoding at least one LDH, and the gene may be exogenous. A polynucleotide encoding LDH may be from bacteria, yeasts, fungi, mammals or reptiles. The polynucleotide may be a polynucleotide that encodes at least one LDH selected from the group consisting of Lactobacillus helveticus, L.bulgaricus, L.johnsonii, L.plantarum, Pelodiscus sinensis japonicus, Ornithorhynchus anatinus, Tursiops truncatus, Rattus norvegicus, Xenopus laevis, and Bos Taurus. An LDH derived from Pelodiscus sinensis japonicus, an LDH derived from Ornithorhynchus anatinus an LDH derived from Tursiops truncatus, and an LDH derived from Rattus norvegicus may each include an amino acid sequence having about 60% or more, about 70% or more, about 80% or more, about 90% or more, about 95% or more, about 96% or more, about 97% or more, about 98% or more, or about 99% or more sequence identity with amino acids of SEQ ID NOS: 3, 4, 5, 6, and 7. For example, a polynucleotide encoding the LDH may be a polynucleotide that encodes an amino acid sequence having about 95% or more sequence identity with an amino acid sequence of SEQ ID NOS: 3, 4, 5, 6, and 7. In some embodiments, a polynucleotide encoding the LDH may include a polynucleotide sequence that encodes an amino acid sequence having about 95% or more sequence identity with an amino acid sequence of SEQ ID NOS: 3, 4, 5, 6, and 7, a polynucleotide sequence of SEQ ID NO: 8, or a polynucleotide sequence of SEQ ID NO: 9.

A polynucleotide encoding the LDH may be included in a vector. Examples of the vector may include a replication origin, a promoter, a LDH-encoding polynucleotide, and a terminator. The replication origin may include a yeast autonomous replication sequence (ARS). The yeast ARS may be stabilized by a yeast centrometric sequence (CEN). The promoter may be selected from the group consisting of a cytochrome c (CYC) promoter, a transcription elongation factor (TEF) promoter, a glycerol-3-phosphate dehydrogenase (GPD) promoter, an alcohol dehydrogenase (ADH) promoter, and a promoter of CCW12 gene. The CYC promoter, the TEF promoter, the GPD promoter, the ADH promoter, and the promoter of CCW12 gene may each have a nucleotide sequence of SEQ ID NOS: 24, 25, 26, and 27. The terminator may be selected from the group consisting of a terminator of a gene encoding a phosphoglycerate kinase 1 (PGK1), a terminator of a gene encoding a cytochrome c 1 (CYC1), a terminator of a gene encoding a galactokinase 1 (GAL1), and a trehalose-6-phosphate synthase 1 (TPS1). The CYC1 terminator may have a nucleotide sequence of SEQ ID NO: 28. The TPS1 terminator may have a nucleotide sequence of SEQ ID NO: 29 or 30. The vector may further include a selection marker. The LDH-encoding polynucleotide may be included in a genome at a specific location of a yeast cell. The specific location of the recombinant yeast cell may include a locus of a gene to be deleted and disrupted, such as pyruvate decarboxylase (PDC) or cytochrome-c oxidoreductase 2 (CYB2). When the LDH-encoding polynucleotide functions to produce an active protein in a cell, the polynucleotide is considered as “functional” in a cell.

The recombinant yeast cell may include a polynucleotide that encodes one LDH or a polynucleotide that encodes multiple LDH copies, e.g., 2 to 10 copies. The yeast cell may include a polynucleotide that encodes multiple LDH copies into, for example, 1 to 8, 1 to 7, 1 to 6, 1 to 5, 1 to 4, or 1 to 3 copies. When the yeast cell includes the polynucleotide encoding multiple LDHs, each polynucleotide may include copies of the same LDH polynucleotide or copies of polynucleotides encoding at least two different LDHs. The multiple copies of the polynucleotide encoding exogenous LDHs may be included in the same locus or multiple loci in a genome of a host cell, and the promoter or terminator of each copy of the polynucleotide may be identical to or different from each other.

Activity of the yeast cell that interrupts production of metabolic products for producing lactate may be inactivated or reduced. Also, the activity of a pathway that catalyzes or assists the flow of metabolic products for producing lactate may be increased.

In the recombinant yeast cell, activity of a polypeptide that converts pyruvate into acetaldehyde, a polypeptide that converts lactate into pyruvate, a polypeptide that converts dihydroxyacetone phosphate (DHAP) into glycerol-3-phosphate, an external mitochondrial NADH dehydrogenase, or a combination thereof may be reduced.

A gene encoding a polypeptide that converts pyruvate into acetaldehyde may be deleted or disrupted. The polypeptide that converts pyruvate into acetaldehyde may be an enzyme that is classified under EC 4.1.1.1. The polypeptide that converts pyruvate to acetaldehyde may be a pyruvate decarboxylase, PDC1, PDC5 or PDC6. The polypeptide that converts pyruvate to acetaldehyde may include an amino acid sequence having a sequence identity of about 60% or more, about 70% or more, about 80% or more, about 90% or more, about 95% or more, about 96% or more, about 97% or more, about 98% or more, or about 99% or more with an amino acid sequence of SEQ ID NO: 10. The gene encoding the polypeptide that converts pyruvate to acetaldehyde may be a polynucleotide that encodes an amino acid sequence having about 95% or more sequence identity with respect to an amino acid sequence of SEQ ID NO: 10, or may have a polynucleotide sequence of SEQ ID NO: 11. For example, the gene may be pdc1.

A gene encoding a polypeptide that converts lactate into pyruvate may be deleted or disrupted. The polypeptide that converts lactate into pyruvate may be a cytochrome c-dependent enzyme. The polypeptide that converts lactate into pyruvate may be an enzyme that is classified under EC 1.1.2.4 that acts on D-lactate or EC 1.1.2.3 that acts on L-lactate. The polypeptide that converts lactate into pyruvate may be lactate:cytochrome c-oxidoreductase, for example, a CYB2 (CAA86721.1), a CYB2A, a CYB2B, a DLD1, a DLD2, or a DLD3. The polypeptide that converts lactate into pyruvate may include an amino acid sequence having about 60% or more, about 70% or more, about 80% or more, about 90% or more, about 95% or more, about 96% or more, about 97% or more, about 98% or more, or about 99% or more sequence identity with an amino acid sequence of SEQ ID NO: 12. The gene encoding the polypeptide that converts lactate into pyruvate may be a polynucleotide sequence that encodes an amino acid sequence having about 95% or more sequence identity with an amino acid sequence of SEQ ID NO: 12, or may include a polynucleotide sequence of SEQ ID NO: 13.

A gene encoding the polypeptide that converts DHAP into glycerol-3-phosphate may be deleted or disrupted. The polypeptide that converts DHAP into glycerol-3-phosphate may be a cytosolic glycerol-3-phosphate dehydrogenase and may be an enzyme that catalyzes reduction of DHAP to glycerol-3-phosphate by using oxidation of NADP to NAD+ or NADP+. The polypeptide may be classified under EC 1.1.1.8. The cytosolic glycerol-3-phosphate dehydrogenase may be GPD1. Also, the polypeptide that converts DHAP into glycerol-3-phosphate may be glycerol-3-phosphate dehydrogenase (quinone) (EC.1.1.5.3) or glycerol-3-phosphate dehydrogenase (NAD(P)+) (EC.1.1.1.94). The glycerol-3-phosphate dehydrogenase (quinone) (EC.1.1.5.3) may be GPD2. The glycerol-3-phosphate dehydrogenase may include an amino acid sequence having about 60% or more, about 70% or more, about 80% or more, about 90% or more, about 95% or more, about 96% or more, about 97% or more, about 98% or more, or about 99% or more sequence identity with an amino acid sequence of SEQ ID NO: 14. A gene encoding the cytosolic glycerol-3-phosphate dehydrogenase may include a polynucleotide sequence encoding an amino acid sequence having a sequence identity of about 95% or more with an amino acid sequence of SEQ ID NO: 14, or may include a polynucleotide sequence of SEQ ID NO: 15.

A gene encoding the polypeptide that converts acetaldehyde into ethanol may be deleted or disrupted. The polypeptide may be an enzyme that catalyzes conversion of acetaldehyde to ethanol. The polypeptide may be classified under EC. 1.1.1.1. The polypeptide may be an enzyme that catalyzes conversion of acetaldehyde to ethanol. The polypeptide may be an alcohol dehydrogenase (Adh), for example, Adh1, Adh2, Adh3, Adh4, AdhS, Adh6, or Adh7. The polypeptide may include an amino acid sequence having about 60% or more, about 70% or more, about 80% or more, about 90% or more, about 95% or more, about 96% or more, about 97% or more, about 98% or more, or about 99% or more sequence identity with an amino acid sequence of SEQ ID NO: 16. The gene encoding the polypeptide may include a polynucleotide sequence encoding an amino acid sequence having about 95% or more of sequence identity with an amino acid sequence of SEQ ID NO: 16, or may include a polynucleotide sequence of SEQ ID NO: 17. For example, the gene may be adh1, adh2, adh3, adh4, adh5, adh6, or adh7.

A gene that encodes an aldehyde dehydrogenase (ALD) may be deleted or disrupted. The aldehyde dehydrogenase may be an enzyme that is classified under EC.1.2.1.4. The aldehyde dehydrogenase may be ALD6, and ALD6 may encode a constitutive cytosolic form of an aldehyde dehydrogenase. ALD6 is activated by Mg2+ and may be NADP specific. The enzyme may be involved in production of acetate. A cytosolic acetyl-CoA may be synthesized from the acetated thus produced. The aldehyde dehydrogenase may include an amino acid sequence having about 60% or more, about 70% or more, about 80% or more, about 90% or more, about 95% or more, about 96% or more, about 97% or more, about 98% or more, or about 99% or more sequence identity with an amino acid sequence of SEQ ID NO: 18. The gene encoding the aldehyde dehydrogenase may include a polynucleotide sequence encoding an amino acid sequence having about 95% or more sequence identity with an amino acid sequence of SEQ ID NO: 18, or may include a polynucleotide sequence of SEQ ID NO: 19. For example, the gene may be ald6.

The recombinant yeast cell may have activity of converting acetaldehyde to acetyl-CoA or may include a gene that encodes a polypeptide that can convert acetaldehyde to acetyl-CoA. In the recombinant yeast cell, the activity of converting acetaldehyde to acetyl-CoA may be increased. The polypeptide converting acetaldehyde to acetyl-CoA may be “acetaldehyde dehydrogenase (acetylating)” or “acetaldehyde:NAD+ oxidoreductase (CoA-acetylating)”. The polypeptide converting acetaldehyde to acetyl-CoA may be classified under EC.1.2.1.10. The polypeptide may catalyze reversible conversion of acetaldehyde+coenzyme A+NAD⁺ to acetyl-CoA+NADH. The polypeptide may be acylating acetaldehyde dehydrogenase (A-ALD). An example of the A-ALD may be an E. coli-derived MhpF or a functional homologue, for example, an E. coli-derived or S. typhimurium-derive EutE, or Pseudomonas sp. CF600-derived dmpF. The polypeptide may be derived from Escherichia coli. The acetaldehyde dehydrogenase gene, mhpF, may be one of units that are constituted of transcription units of mhpA, mhpB, mhpC, mhpD, mhpE, and mhpF. In other microorganisms, MhpE and MhpF constitute one complex, but in E. Coli, MhpF may exist alone and has activity. The polypeptide converting acetaldehyde to acetyl-CoA may include an amino acid sequence having about 60% or more, about 70% or more, about 80% or more, about 90% or more, about 95% or more, about 96% or more, about 97% or more, about 98% or more, or about 99% or more sequence identity with an amino acid sequence of SEQ ID NO: 20. For example, the MhpF may have an amino acid sequence of SEQ ID NO: 20. A gene that encodes the polypeptide may be a polynucleotide that encodes a protein having about 95% or more sequence identity with an amino acid sequence of SEQ ID NO: 20, or may be a polynucleotide having about 95% or more sequence identity with a polynucleotide sequence of SEQ ID NO: 21. For example, in the gene, codons may be substituted so that the polypeptide derived from E. coli may suit the usage in the yeast cell. Here, modification of the gene may be performed by the substitution within a range that does not change sequences of the polypeptide. For example, the gene modified to suit the usage in the yeast cell may have a polynucleotide sequence of SEQ ID NO: 22.

Also, in the recombinant yeast cell according to an exemplary embodiment, activity of ERG5 is increased; a gene that encodes a polypeptide converting pyruvate to acetaldehyde, a gene that encodes a polypeptide converting lactate to pyruvate, a gene that encodes a polypeptide converting DHAP to glycerol-3-phosphate, a gene that encodes an external mitochondrial NADA dehydrogenase, a gene that encodes a polypeptide converting acetaldehyde to ethanol, a gene that encodes aldehyde dehydrogenase, or a combination thereof is deleted or disrupted; and a gene that encodes a polypeptide converting pyruvate to lactate, a gene that encodes a polypeptide converting acetaldehyde to acetyl-CoA, or a combination thereof is introduced to the yeast cell. The yeast cell may be Saccharomyces cerevisiae.

According to another embodiment, a composition for producing lactate is provided, wherein the composition includes a yeast cell. The yeast cell is the same as described above.

According to another embodiment, a method of producing lactate is provided, wherein the method includes culturing a yeast cell. The yeast cell is the same as described above.

The culturing of the yeast cell may be performed in a suitable medium under suitable culturing conditions known in the art. One of ordinary skill in the art may suitably change a culture medium and culturing conditions according to the microorganism selected. A culturing method may be batch culturing, continuous culturing, or fed-batch culturing. The yeast cell is the same as described above.

The culture medium may include various carbon sources, nitrogen sources, and trace elements.

The carbon source may be, for example, carbohydrate such as glucose, sucrose, lactose, fructose, maltose, starch, or cellulose; fat such as soybean oil, sunflower oil, castor oil, or coconut oil; fatty acid such as palmitic acid, stearic acid, or linoleic acid; alcohol such as glycerol or ethanol; organic acid such as acetic acid, and/or a combination thereof. The culturing may be performed by having glucose as the carbon source. The nitrogen source may be an organic nitrogen source such as peptone, yeast extract, beef stock, malt extract, corn steep liquor (CSL), or soybean flour, or an inorganic nitrogen source such as urea, ammonium sulfate, ammonium chloride, ammonium phosphate, ammonium carbonate, and ammonium nitrate, or a combination thereof. The culture medium is a supply source of phosphorus and may include, for example, potassium dihydrogen phosphate, dipotassium phosphate, and corresponding sodium-containing salt thereof, and a metal salt such as magnesium sulfate or iron sulfate. Also, amino acid, vitamin, a suitable precursor, or the like may be included in the culture medium. The culture medium or individual component may be added to a culture medium solution in a batch or continuous manner.

Also, pH of the culture medium solution may be adjusted by adding a compound such as ammonium hydroxide, potassium hydroxide, ammonia, phosphoric acid, and sulfuric acid to the culture medium solution by using a suitable method during the culturing process. Also, an antifoaming agent such as fatty acid polyglycol ester may be used during the culturing process to inhibit the generation of bubbles.

The yeast cell may be cultured under an aerobic, microaerobic, or anaerobic condition. In some embodiments, the microaerobic condition refers to a culturing condition in which oxygen is dissolved in the medium at a lower level than that in the atmosphere. For example, the lower oxygen concentration level may be about 0.1% to about 10%, about 1% to about 9%, about 2% to about 8%, about 3% to about 7%, or about 4% to about 6% of a saturated dissolved oxygen in the atmosphere. Also, the microaerobic condition may include maintaining a dissolved oxygen (DO) concentration of the medium in a range of about 0.9 ppm to about 3.6 ppm, for example, about 0.9 ppm to about 3.6 ppm. A temperature for the culturing may be in a range of, for example, about 20° C. to about 45° C. or about 25° C. to about 45° C. A period of time for the culturing may be continued until a desired amount of lactate is obtained. The method of producing lactate may include collecting or isolating lactate from the culture.

The collecting of lactate from the culture may be performed by using a separation and purification method known in the art. The collecting of lactate may be performed by centrifugation, ion-exchange chromatography, filtration, precipitation, extraction, distillation, or combination thereof. For example, the culture may be centrifuged to separate biomass, and a supernatant thus obtained may be separated by ion-exchange chromatography.

The yeast cell according to an aspect of the present disclosure may be produced at a high concentration and a high yield.

Lactate may be produced at a high concentration and a high yield by using the method of producing lactate according to another embodiment.

Hereinafter, the present disclosure will be described in further detail with reference to the following examples. These examples are for illustrative purposes only and are not intended to limit the scope of the present disclosure.

### EXAMPLE 1

### Manufacture of Yeast Cell with Improved Capability of Producing Lactate

1. Manufacture of Yeast Cell with Improved Capability of Producing Lactate

In order to improve capability of producing lactate in S. cerevisiae CEN.PK2-1D, genes of pyruvate decarboxylase 1 (PDC1) and alcohol dehydrogease 1 (ADH1), which are enzymes involved in a pathway to detour the metabolic product flow to avoid a flow of lactate production, where the pathway is a pathway from pyruvate to ethanol, were deleted. PDC1 is an enzyme that catalyzes conversion of pyruvate to acetaldehyde and CO₂. ADH1 is an enzyme that catalyzes conversion of acetaldehyde to ethanol.

Here, a lactate dehydrogenase (ldh) gene was introduced to the yeast cell at the same time the pdc1 gene and the adh1 gene were deleted. LDH is an enzyme that catalyzes conversion of pyruvate to lactate.

Also, an L-lactate cytochrome-c oxidoreductase (cyb2) gene that catalyzes conversion of lactate to pyruvate was deleted. Here, an ldh gene was introduced to the yeast cell at the same time the cyb2 gene was deleted.

Also, in order to enhance the metabolic flow to pyruvate in a glycolysis process, a glycerol-3-phosphate dehydrogenase 1 (gpd1) gene having activity of converting dihydroxy acetone phosphate (DHAP) to glycerol-3-phosphate (G3P) was deleted. GPD1 converts NADH to NAD+ at the same time converting DHAP to G3P. Here, an ldh gene was introduced to the yeast cell at the same time the gpd1 gene was deleted.

Also, a gene that encodes MhpF (acetaldehyde dehydrogenase(acylating)) derived from E. coli was introduced to S. cerevisiae CEN.PK2-1D. MhpF may be classified under EC.1.2.1.10. MhpF may catalyze conversion of acetaldehyde to acetyl-CoA. MhpF may use NAD+ and a coenzyme A. MhpF may be the last enzyme of a meta-cleavage pathway for cleavage of 3-HPP. A MhpF gene may be introduced to a site of an ald6 gene that encodes an aldehyde dehydrogenase 6 (ALD6), so as to delete the ald6 gene. ALD6 may encode a constitutive cytosolic form of an aldehyde dehydrogenase. ALD6 may be activated by Mg²⁺ and may be NADP specific. The enzyme may be involved in production of acetate. A cytosolic acetyl-CoA may be synthesized from acetate.

(1) Manufacture of S. cerevisiae CEN.PK2-1D (Δ pdc1::ldh)

(1.1) Manufacture of Vector to Introduce Ldh While Deleting pdc1

In order to block a pathway from pyruvate to ethanol through acetaldehyde in S. cerevisiae CEN.PK2-1D, a gene that encodes a pyruvate decarboxylase1 (pdc1) was deleted. In order to express an Ldh derived from Pelodiscus sinensis japonicus while deleting a pdc1 gene at the same time, a pdc1 gene was deleted by substituting the pdc1 gene with ‘an ldh cassette’. As used herein, unless stated otherwise, the term “cassette” refers to a unit sequence from which a protein may be expressed, where cassette includes operably linked promoters, coding sequences, and terminators.

In particular, to manufacture a vector including an ‘ldh cassette’, PCR was performed using a genomic DNA of S. cerevisiae as a template and a primer pair of SEQ ID NOS: 31 and 32 as primers to obtain a CCW12 promoter sequence (SEQ ID NO: 27) and an ‘ldh gene (SEQ ID NO: 9)’. The CCW12 promoter sequence (SEQ ID NO: 27) and the ldh gene (SEQ ID NO: 9) were each digested with SacI/XbaI and BamHI/SalI, and linked to a pRS416 vector (ATCC87521), which was digested with the same enzyme. The pRS416 vector is a yeast centromere shuttle plasmid with a T7 promoter, ampicillin resistance in bacteria, a URA3 cassette in yeast (a selection marker), and a restriction enzyme cloning site. Next, PCR was performed on the vector thus obtained using a pCEP4 plasmid (invitrogen, Cat. no. V044-50) as a template and a primer pair of SEQ ID NOS: 33 and 34 as primers to obtain amplification product, which was a ‘hygromycin B phosphotransferase (HPH) cassette’ sequence (SEQ ID NO: 35). The HPH cassette sequence was digested with SacI, and then linked to the vector digested with the same enzyme to prepare a vector p416-ldh-HPH including the ‘ldh cassette’. The pCEP4 plasmid is an episomal mammalian expression vector that uses cytomegalovirus (CMV) immediate early enhance/promoter for high level transcription of recombinant genes inserted into the multiple cloning site. The pCEP4 also carries the hygromycin B resistance gene for stable selection in transfected cells. Here, an ‘ldh cassette’ includes an ldh gene and its regulatory region, and thus the ldh cassette refers to a region that allows the ldh gene to be expressed. The ldh gene was transcripted under the control of the CCW 12 promoter. Also, the ‘HPH cassette’ includes a hygromycin B resistance gene and its regulatory region, and thus the HPH cassette refers to a region that allows the hygromycin B resistant gene to be expressed.

In order to prepare a pdc1 deletion cassette, PCR was performed using p416-ldh-HPH as a template and a primer set of SEQ ID NOS: 36 and 37 as primers to prepare a ldh gene fragment and a pUC57-Ura3HA vector(DNA2.0 Inc.; SEQ ID NO: 38). The ldh gene fragment and pUC57-Ura3HA vector were each digested with SacI and then linked to each other to prepare a pUC-uraHA-ldh vector. PCR was performed using sequences of SEQ ID NOS: 39 and 40 having the homologous sequence with the pdc1 gene as primers to amplify the pdc1 deletion cassette from the pUC-uraHA-ldh vector. 1 to 41 of SEQ ID NO: 39 and 1 to 44 of SEQ ID NO: 40 denote sites of homologous recombination with the homologous chromosomes of S. cerevisea and substituted with the pdc1 gene.

(1.2) Manufacture of S. cerevisiae CEN.PK2-1D (Δ pdc1::ldh)

The pdc1 deletion cassette prepared in (1.1) was introduced to S. cerevisiae (CEN.PK2-1D, EUROSCARF accession number: 30000B). The introduction of the pdc1 deletion cassette was performed by general heat shock transformation. After the transformation, the cells were cultured in a uracil drop out medium to allow the pdc1 ORF on the chromosome to be substituted with the cassette.

Then, PCR was performed using a genome of the cells as a template and a primer set of SEQ ID NOS: 41 and 42 as primers on the cells thus obtained to confirm deletion of pdc1. Therefore, deletion of the pdc1 gene and introduction of the ldh gene were confirmed. As a result, S. cerevisiae CEN.PK2-1D (Δ pdc1::ldh) was manufactured.

(2) Manufacture of S. cerevisiae CEN.PK2-1D (Δ pdc1::ldh, Δ cyb2::ldh)

(2.1) Manufacture of Vector to Delete cyb2

In order to block a pathway from lactate to pyruvate, a cyb gene was deleted from the S. cerevisiae CEN.PK2-1D (Δ pdc1::ldh) prepared in (1).

In particular, PCR was performed using the pUC-uraHA-ldh vector prepared in (1.1) as a template and cyb2 homologous recombinant sequences of SEQ ID NOS: 43 and 44 as primers to obtain a cyb2 deletion cassette. 1 to 45 of SEQ ID NO: 43 and 1 to 45 of SEQ ID NO: 44 denote sites of homologous recombination with chromosomes of S. cerevisea and substituted with the cyb2 gene.

(2.2) Manufacture of S. cerevisiae CEN.PK2-1D (Δ pdc1::ldh, Δ cyb2::ldh)

The cyb2 deletion cassette prepared in (2.1) was introduced to S. cerevisiae CEN.PK2-1D (Δ pdc1::ldh). The introduction of the cyb2 deletion cassette was performed by general heat shock transformation. After the transformation, the cells were cultured in a uracil drop out medium to allow the cyb2 ORF on the chromosome to be substituted with the cassette.

Then, PCR was performed using a genome of the cells as a template and a primer set of SEQ ID NOS: 45 and 46 as primers on the strain thus obtained to confirm deletion of cyb2. As a result, S. cerevisiae CEN.PK2-1D (Δ pdc1::ldh, Δ cyb2::ldh) was manufactured.

(3) Manufacture of S. cerevisiae CEN.PK2-1D (Δ pdc1::ldh, Δ cyb2::ldh, Δ gpd1::ldh)

(3.1) Manufacture of Vector to Delete gpd1

In order to block a pathway from DHAP to glycerol-3-phosphate, a gene encoding a glycerol-3-phosphate dehydrogenase 1 (gpd1) was deleted from the S. cerevisiae CEN.PK2-1D (Δ pdc1::ldh, Δ cyb2::ldh) prepared in (2).

In particular, PCR was performed using the pUC-uraHA-ldh vector prepared in (1.1) as a template and gpd1 homologous recombinant sequences of SEQ ID NOS: 47 and 48 as primers to obtain a gpd1 deletion cassette. 1 to 50 of SEQ ID NO: 47 and 1 to 50 of SEQ ID NO: 48 denote sites of homologous recombination with chromosomes of S. cerevisiae and substituted with the gpd1 gene.

(3.2) Manufacture of S. cerevisiae CEN.PK2-1D(Δ pdc1::ldh, Δ cyb2::ldh, Δ gpd1::ldh)

The gpd1 deletion cassette prepared in (3.1) was introduced to S. cerevisiae CEN.PK2-1D(Δ pdc1::ldh, Δ cyb2::ldh) prepared in (2). The introduction of the pdc1 deletion cassette was performed by general heat shock transformation. After the transformation, the cells were cultured in a uracil drop out medium to allow the gdp1 ORF on the chromosome to be substituted with the cassette.

Then, PCR was performed using a genome of the cells as a template and a primer set of SEQ ID NOS: 49 and 50 as primers on the strain thus obtained to confirm deletion of gpd1. As a result, S. cerevisiae CEN.PK2-1D(Δ pdc1::ldh, Δ cyb2::ldh, Δ gpd1::ldh) was manufactured.

S. cerevisiae CEN.PK2-1D(Δ pdc1::ldh, Δ cyb2::ldh, Δ gpd1::ldh) has been deposited to Korean Collection for Type Cultures according to the Budapest Treaty (KCTC Accession No. 12415BP).

(4) Manufacture of S. cerevisiae CEN.PK2-1D(Δ pdc1::ldh, Δ cyb2::ldh, Δ gpd1::ldh, Δ adh1::ldh)

(4.1) Manufacture of Vector to Delete adh 1

In order to block a pathway from acetaldehyde to ethanol, a gene encoding a alcohol dehydrogenase (adh1) was deleted from the S. cerevisiae CEN.PK2-1D (Δ pdc1::ldh, Δ cyb2::ldh, Δ gpd1::ldh) prepared in (3). In order to express Ldh at the same time deleting the adh1 gene, the adh1 gene was substituted with a ldh-HPH cassette and deleted.

PCR was performed using the p416-ldh-HPH vector prepared in (1.1) as a template and adh1 homologous recombinant sequences of SEQ ID NOS: 51 and 52 as primers to obtain an adh1 deletion cassette. 1 to 51 of SEQ ID NO: 51 and 1 to 51 of SEQ ID NO: 52 denote sites of homologous recombination with chromosomes of S. cerevisea and substituted with the adh1 gene.

(4.2) Manufacture of S. cerevisiae CEN.PK2-1D(Δ pdc1::ldh, Δ cyb2::ldh, Δ gpd1::ldh, Δ adh1::ldh)

The adh1 deletion cassette prepared in (4.1) was introduced to S. cerevisiae CEN.PK2-1D(Δ pdc1::ldh, Δ cyb2::ldh, Δ gpd1::ldh) prepared in (3). The introduction of the adh1 deletion cassette was performed by general heat shock transformation. After the transformation, the cells were cultured in the presence of a selection marker, hygromycin B, to allow the adh1 ORF on the chromosome to be substituted with the cassette.

Then, PCR was performed using a genome of the cells as a template and a primer set of SEQ ID NOS: 53 and 54 as primers on the cells thus obtained to confirm deletion of adh1 and introduction of the ldh gene. As a result, S. cerevisiae CEN.PK2-1D(Δ pdc1::ldh, Δ cyb2::ldh, Δ gpd1::ldh, Δ adh1::ldh) was manufactured.

(5) Manufacture of S. cerevisiae CEN.PK2-1D(Δ pdc1::ldh, Δ cyb2::ldh, Δ gpd1::ldh, Δ adh1::ldh, Δ ald6::mhpF)

(5.1) Manufacture and Introduction of Vector to Introduce mhpF

In order to enhance a pathway of converting acetaldehyde to acetyl-CoA in S. cerevisiae CEN.PK2-1D(Δ pdc1::ldh, Δ cyb2::ldh, Δ gpd1::ldh, Δ adh1::ldh) prepared in (4), a MhpF gene was introduced to an ald6 gene site.

In particular, the MhpF gene was prepared by obtaining a S.cerevisiae codon-optimized nucleotide sequence based on a MhpF gene derived from E. coli and synthesizing the sequence (DNA2.0 Inc; SEQ ID NO: 22). The MhpF gene thus obtained and an ‘HIS3 cassette’ were each linked to a ‘pUC19 vector’ (NEB, N3041) by using a restriction enzyme, Sall, to prepare a pUC19-His-MhpF vector (SEQ ID NO: 55). The HIS3 cassette was an amplification product obtained through amplification by performing PCR using pRS413 (ATCC8758) as a template and a primer set of SEQ ID NOS: 56 and 57 as primers. In pUC19-His-MhpF vector, the mhpF was expressed under the control of a GPD promoter (SEQ ID NO: 25).

PCR was performed using the pUC19-His-MhpF thus prepared and promoter-linked ald6 homologous recombinant sequences of SEQ ID NOS: 58 and 59 as primers to obtain a mhpF insertion cassette. 1 to 44 of SEQ ID NO: 58 and 1 to 45 of SEQ ID NO: 59 denote sites of homologous recombination with chromosomes of S. cerevisea and substituted with the ald6 gene.

(5.2) Manufacture of S. cerevisiae CEN.PK2-1D(Δ pdc1::ldh, Δ cyb2::ldh, Δ gpd1::ldh, Δ adh1::ldh, Δ ald6::mhpF)

The mhpF insertion cassette prepared in (5.1) was introduced to S. cerevisiae CEN.PK2-1D(Δ pdc1::ldh, Δ cyb2::ldh, Δ gpd1::ldh, Δ adh1::ldh) prepared in (4). The introduction of the mhpF insertion cassette was performed by general heat shock transformation. After the transformation, the cells were cultured in a histidine drop out medium (including 6.7 g/L of yeast nitrogen base without amino acids (Sigma-Aldrich: cat. no. Y0626), 1.9 g/L of yeast synthetic drop-out without histidine (Sigma-Aldrich: cat. no. Y1751), and 2 (w/v) % glucose) to allow the ald6 ORF on the chromosome to be substituted with the cassette.

In order to confirm deletion of the ald6 gene and introduction of the mhpF gene in the strain obtained as the result, PCR was performed using a genome of the cells as a template and a primer set of SEQ ID NOS: 60 and 61 as primers. As a result, S. cerevisiae CEN.PK2-1D (Δ pdc1::ldh, Δ cyb2::ldh, Δ gpd1::ldh, Δ adh1::ldh, Δ ald6::mhpF) was manufactured.

### EXAMPLE 2

### Manufacture of EFG5 gene overexpressed S. cerevisiae

2.1 Manufacture of vector for Overexpression of ERG5

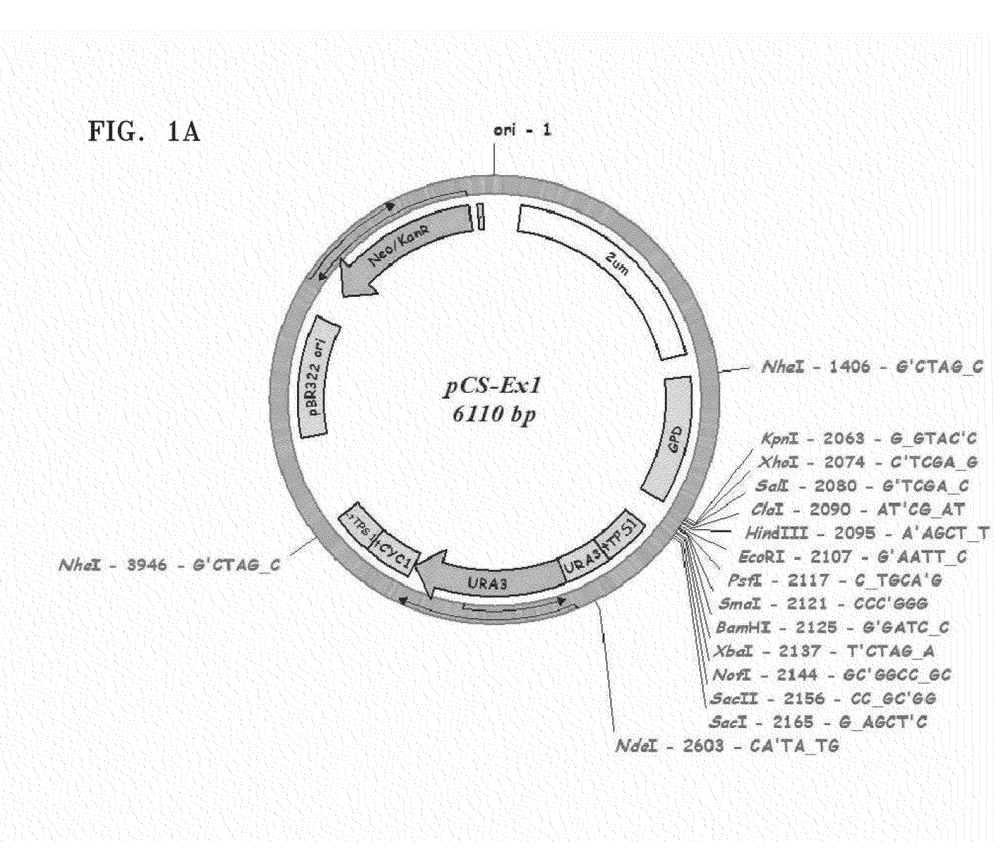

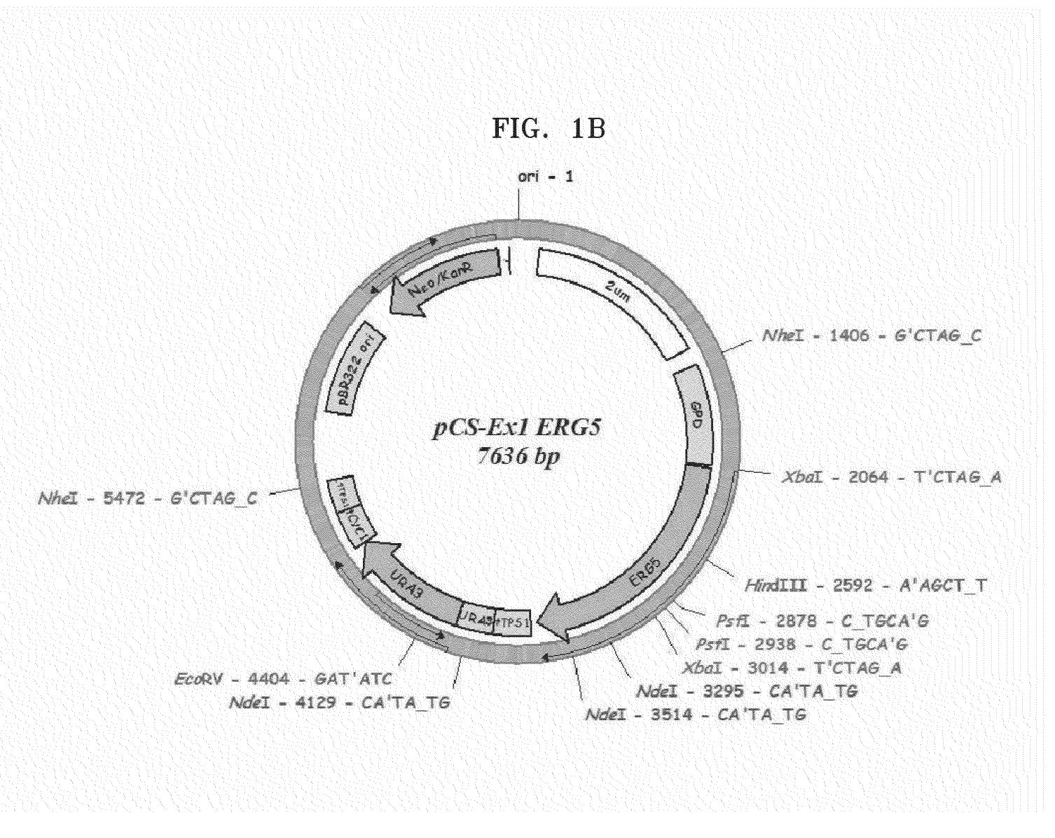

In order to overexpress an ERG5 gene, a coding site of ERG5 was amplified from a genomic DNA of a S. cerevisiae CEN.PK2-1D strain by performing PCR using a primer set of SEQ ID NOS: 62 and 63, and the amplification product was linked to a pCS-Ex1 vector, which was digested with Kpnl and Sac!, by using an infusion cloning kit to prepare a pCS-Ex1 ERG5 vector. The ERG5 gene in the vector may be transcribed under the control of a GPD promoter. FIG. 1A illustrates a pCS-Ex1 vector. FIG. 1B illustrates a pCS-Ex1 ERG5 vector. In the pCS-Ex1 ERG5, ERG5 may be expressed under the control of the GPD promoter (SEQ ID NO: 25) which includes a Kozak consensus sequence (AAACA).

2.2 Introduction of ERG5 Gene to S. cerevisiae CEN.PK2-1D (Δ pdc1::ldh, Δ cyb2, Δ gpd1, Δ adh1::ldh, Δ ald6::mhpF)

In order to overexpress an ERG5 gene, an ERG5 expression cassette was introduced to a pdc6 gene site by using the pCS-Ex1 ERG5 vector prepared in 2.1. In particular, a cassette was amplified by performing PCR using a primer set of SEQ ID NOS: 64 and 65 from the pCS-Ex1 ERG5 vector, and the cassette was introduced by using a heat shock transformation method. After the transformation, the cells were spread on an agar plate without uracil, which is a selection marker, cultured at 30° C. to confirm introduction of the cassette to a chromosome. In regard to the strain obtained as the result, PCR was performed using a genome of the obtained cells as a template and a primer set of SEQ ID NOS: 66 and 67 as primers to confirm deletion of a PDC6 gene and introduction of an ERG5 gene. As a result, S. cerevisiae CEN.PK2-1D (Δ pdc1::ldh, Δ cyb2::ldh, Δ gpd1::ldh, Δ adh1::ldh, Δ ald6::mhpF, Δ pdc6::ERG5) was manufactured.

### EXAMPLE 3

### Measure of Acid Resistance by Using ERG5 Gene Overexpressed Strain

An ERG5 gene was introduced to a yeast cell and overexpressed to confirm influence of the overexpression on acid resistance of the yeast cell.

S. cerevisiae CEN.PK2-1D (Δ pdc1 ::ldh, Δ cyb2::ldh, Δ gpd1::ldh, Δ adh1::ldh, Δ ald6::mhpF) strain prepared in Example 1, as a control group, and S. cerevisiae CEN.PK2-1D (Δ pdc1::ldh, Δ cyb2::ldh, Δ gpd1::ldh, Δ adh1::ldh, Δ ald6::mhpF, Δ pdc6::ERG5) strain prepared in Example 2, as an experiment group, were each spread on a YPD agar medium (including 20 g/L of Bacto Peptone, 10 g/L of yeast extract, 20 g/L of D-glucose, 20 g/L of Bacto agar) and incubated for 48 hours or more at 30° C., and then, a colony obtained therefrom was inoculated in about 2 ml of a YPD liquid medium (including 20 g/L of Bacto Peptone, 10 g/L of yeast extract, and 20 g/L of D-glucose) and cultured for about 24 hours at 30° C. under an aerobic condition while stirring at a rate of about 230 rpm. A concentration of the cultured cells was measured by using absorbance at 600 nm, and each sample was prepared by serially diluting the cultured cells in 10 uL of sterilized water to have 10, 10², 10³, or 10⁴ cells in the sample. The samples were inoculated to a medium including lactic acid to confirm survival and growth of the yeast cell.

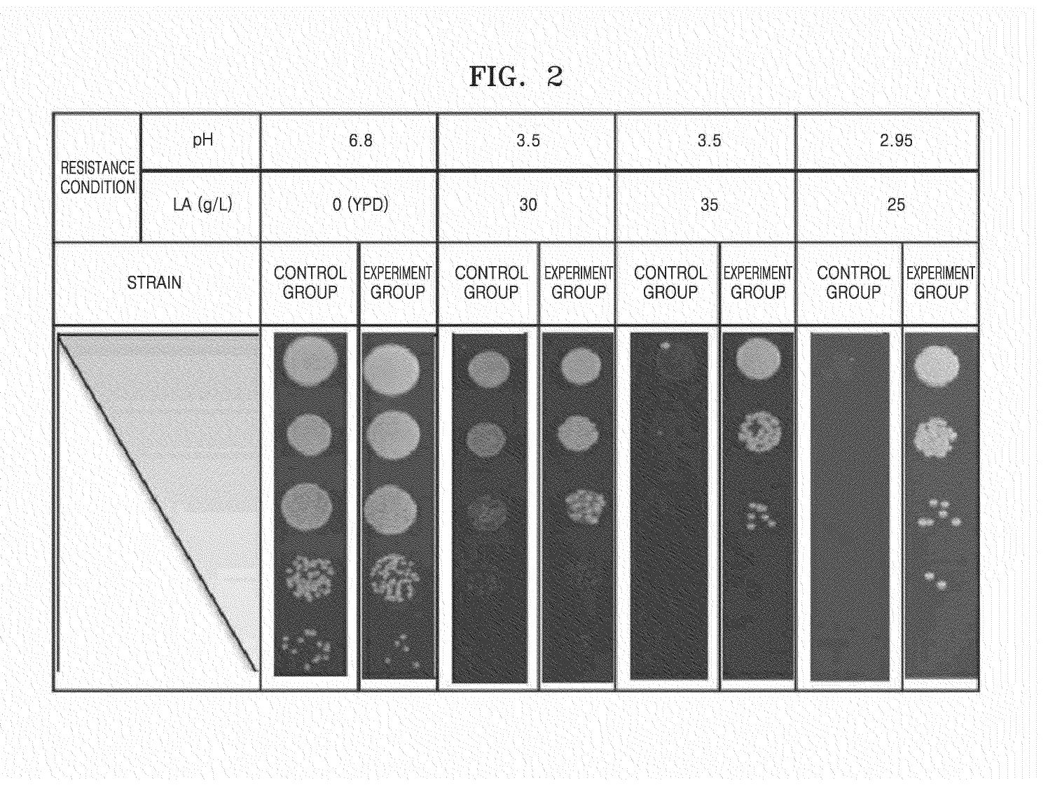

FIG. 2 shows the result of culturing various yeast cells on an YPD acid medium including 25 g/L of lactic acid and having pH adjusted to 2.95. As shown in FIG. 2, the ERG5 overexpressed S. cerevisiae CEN.PK2-1D(Δ pdc1::ldh, Δ cyb2::ldh, Δ gpd1::ldh, Δ adh1::ldh, Δ ald6::mhpF, Δ pdc6::ERG5) strain was well-grown in an acid medium including lactic acid at a pH of 2.95 compared to the control group, i.e, the number of colonies of the ERG5 overexpressed S. cerevisiae was more than those of the control group.

In this regard, the ERG5 gene-overexpressed yeast strain grows better when an acidity of the medium increases compared to the control group. That is, the ERG5 gene-overexpressed yeast strain is resistant to acid. Also, effect of acid resistance confirms that the yeast is acid resistant regardless of an organic acid produced by the yeast.

### EXAMPLE 4

### Production of Lactate by Using ERG5 Gene Overexpressed Strain

An ERG5 gene was introduced to a yeast cell and overexpressed to confirm influence of the overexpression on glucose uptake and lactate production of the yeast cell.

S. cerevisiae CEN.PK2-1D (Δ pdc1::ldh, Δ cyb2::ldh, Δ gpd1::ldh, Δ adh1::ldh, Δ ald6::mhpF) strain prepared in Example 1, as a control group, and S. cerevisiae CEN.PK2-1D (Δ pdc1::ldh, Δ cyb2::ldh, Δ gpd1::ldh, Δ adh1::ldh, Δ ald6::mhpF, Δ pdc6::ERG5) strain prepared in Example 2, as an experiment group, were each spread on a YPD agar medium (including 20 g/L of Bacto Peptone, 10 g/L of yeast extract, 20 g/L of D-glucose, 20 g/L of Bacto agar) and incubated for 48 hours or more at 30° C., and then, a colony obtained therefrom was inoculated in about 2 ml of a YPD liquid medium (including 20 g/L of Bacto Peptone, 10 g/L of yeast extract, and 20 g/L of D-glucose) and cultured for about 24 hours at 30° C. under an aerobic condition while stirring at a rate of about 230 rpm. The cultured cells were inoculated to a fresh YPD liquid medium (including 20 g/L of Bacto Peptone, 10 g/L of yeast extract, and 20 g/L of D-glucose) to have a final cell concentration of 10⁶ cells/ml, and cultured for about 8 hours at 30° C. under an aerobic condition while stirring at a rate of about 230 rpm to culture the cells so that a growth stage of the cell reach an exponential growth stage. The cultured cells were inoculated into a D-glucose-containing-YPD liquid medium (including 20 g/L of Bacto Peptone, 10 g/L of yeast extract, and 80 g/L of D-glucose) to have a final cell concentration of 4×10⁷ cells/ml, and allowed to produce lactic acid under a fermentation condition at 30° C. for 72 hours under a microaerobic condition of 2.5% oxygen content.

The fermentation condition included maintaining a temperature at 30° C., an atmospheric oxygen content of 2.5%, a humidity of 95% or more, and stirring at a rate of 200 rpm or higher. Samples were periodically obtained so that the oxygen content was maintained the same during the fermentation, and the obtained samples were centrifuged for about 1 minute at a rate of about 13,000 rpm. Then, the supernatant of the sample was filter-sterilized to obtain a culture solution from which the strain was completely removed, and concentrations of lactate and glucose in the culture solution were analyzed by using HPLC.

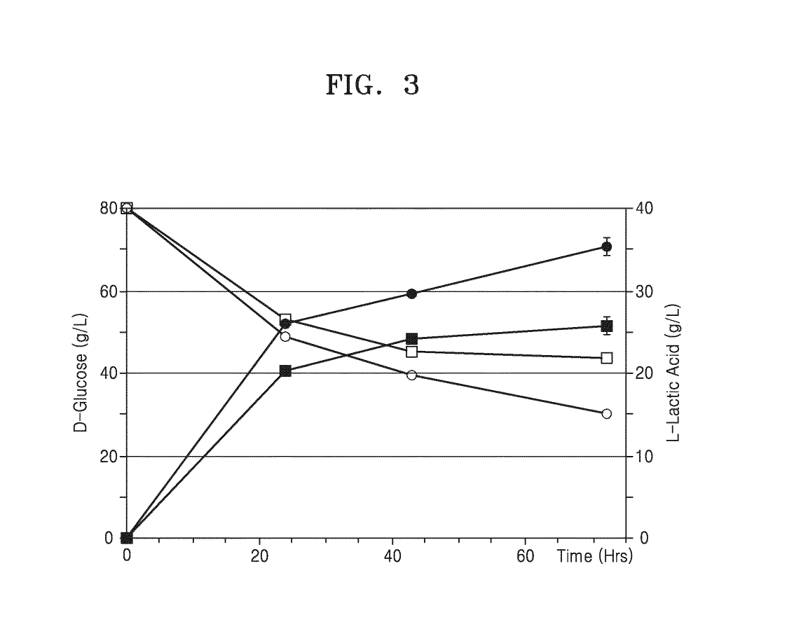

FIG. 3 shows the concentrations of lactate and glucose of the ERG5 gene overexpressed yeast cell and its control group. As shown in FIG. 3, the ERG5 gene overexpressed S. cerevisiae CEN.PK2-1D (Δ pdc1::ldh, Δ cyb2::ldh, Δ gpd1::ldh, Δ adh1::ldh, Δ ald6::mhpF, Δ pdc6::ERG5) strain had higher lactate production and more glucose uptake than those of the S. cerevisiae CEN.PK2-1D (Δ pdc1::ldh, Δ cyb2::ldh, Δ gpd1::ldh, Δ adh1::ldh, Δ ald6::mhpF) strain, in which ERG5 gene was not overexpressed. In particular, in 72 hours after the incubation, lactate production of the ERG5 overexpressed strain increased from 25.8 g/L to 35.3 g/L, and a glucose specific productivity yield increased from 71.2 g/g % to 71.3 g/g %. In this regard, it may be known that lactate production increased in the ERG5 gene overexpressed strain. Also, as compared to the strain in which ERG5 gene is not overexpressed, the ERG5 gene overexpressed strain has a higher capability of producing lactic acid and has improved resistance to the produced lactic acid, and thus the ERG5 gene overexpressed strain may have a higher capability of producing lactic acid and resistance to the produced lactic acid after a long period of culturing time.

It should be understood that the exemplary embodiments described therein should be considered in a descriptive sense only and not for purposes of limitation. Descriptions of features or aspects within each embodiment should typically be considered as available for other similar features or aspects in other embodiments.

While one or more embodiments of the present disclosure have been described with reference to the figures, it will be understood by those of ordinary skill in the art that various changes in form and details may be made therein without departing from the spirit and scope of the present invention as defined by the following claims. All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A recombinant acid-resistant yeast cell comprising increased ERG5 activity compared to ERG5 activity of a parent cell thereof, wherein the yeast cell comprises a genetic modification that increases activity of the ERG5.

2. The recombinant yeast cell of claim 1 comprising increased expression of a polynucleotide encoding an ERG5 polypeptide, compared to ERG5 expression in the parent cell.

3. The recombinant yeast cell of claim 1 comprising an exogenous polynucleotide encoding ERG5.

4. The recombinant yeast cell of claim 1 comprising an increased copy number of a gene encoding ERG5 or a modification of an expression regulatory sequence of the gene encoding ERG5 as compared to a parent cell thereof, thereby increasing ERG5 activity in the recombinant yeast cell compared to the parent cell.

5. The recombinant yeast cell of claim 4 comprising an exogenous gene encoding ERG5 or amplification of an endogenous gene encoding ERG5, thereby increasing the copy number of a gene encoding ERG5 in the recombinant yeast cell.

6. The recombinant yeast cell of claim 1, wherein the increased activity of ERG5 is caused by mutation of the gene encoding ERG5.

7. The recombinant yeast cell of claim 1, wherein ERG5 has about 60% or more sequence identity with an amino acid sequence of SEQ ID NO: 1.

8. The recombinant yeast cell of claim 1, wherein the polynucleotide encoding ERG5 has about 95% or more sequence identity with a polynucleotide sequence of SEQ ID NO: 2.

9. The recombinant yeast cell of claim 1, wherein the recombinant yeast cell is selected from the group consisting of the Saccharomyces genus, Kluyveromyces genus, Candida genus, Pichia genus, Issatchenkia genus, Debaryomyces genus, Zygosaccharomyces genus, Shizosaccharomyces genus, and Saccharomycopsis genus.

10. The recombinant yeast cell of claim 1, wherein the recombinant yeast cell is Saccharomyces cerevisiae.

11. The recombinant yeast cell of claim 1, wherein the recombinant yeast cell produces lactate under microaerobic or anaerobic conditions.

12. The recombinant yeast cell of claim 1 comprising a polynucleotide that encodes a polypeptide that converts pyruvate to lactate.

13. The recombinant yeast cell of claim 12, wherein the polypeptide which can convert pyruvate to lactate comprises an amino acid sequence having about 95% or more sequence identity with an amino acid sequence of SEQ ID NO: 3.

14. The recombinant yeast cell of claim 1, wherein activity of a polypeptide converting pyruvate to acetaldehyde, a polypeptide converting lactate to pyruvate, a polypeptide converting dehydroxyacetone phosphate (DHAP) to glycerol-3-phosphate, a polypeptide converting acetaldehyde to ethanol, an aldehyde dehydrogenase, or a combination thereof is reduced in comparison to the respective activities of the parent cell of the recombinant yeast cell.

15. The recombinant yeast cell of claim 1, further comprising a deletion or disruption of a gene that encodes a polypeptide that converts pyruvate to acetaldehyde, a gene that encodes a polypeptide that converts lactate to pyruvate, a gene that encodes a polypeptide that converts dehydroxyacetone phosphate (DHAP) to glycerol-3-phosphate, a gene that encodes a polypeptide that converts acetaldehyde to ethanol, a gene that encodes an aldehyde dehydrogenase, or a combination thereof.

16. The recombinant yeast cell of claim 1 comprising a gene that encodes a polypeptide that converts acetaldehyde to acetyl-CoA.

17. A method of producing lactate, the method comprising culturing the recombinant yeast cell of claim 1, wherein the yeast cell produces lactate in a culture.

18. The method of claim 17 comprising collecting lactate from the culture.

19. The method of claim 17, wherein the yeast cell is cultured at a pH of ab out 2 to about 7.

20. A method of increasing the acid resistance and/or lactate production of a yeast cell, comprising introducing an exogenous polynucleotide encoding ERG5 into the yeast cell, or increasing the copy number of an endogenous gene encoding ERG5 in the yeast cell.


SEQUENCE LISTING (67 sequences)

SEQ ID NO: 1 - protein, 538 aa, Saccharomyces cerevisiae
MSSVAENIIQHATHNSTLHQLAKDQPSVGVTTAFSILDTLKSMSYLKIFATLICILLVWDQVAYQIKKGSIAGPKFKFWPIIGPFLESLDPKFEEYKAKWASGPLSCVSIFHKFVVIASTRDLARKILQSSKFVKPCVVDVAVKILRPCNWVFLDGKAHTDYRKSLNGLFTKQALAQYLPSLEQIMDKYMDKFVRLSKENNYEPQVFFHEMREILCALSLNSFCGNYITEDQVRKIADDYYLVTAALELVNFPIIIPYTKTWYGKKTADMAMKIFENCAQMAKDHIAAGGKPVCVMDAWCKLMHDAKNSNDDDSRIYHREFTNKEISEAVFTFLFASQDASSSLACWLFQIVADRPDVLAKIREEQLAVRNNDMSTELNLDLIEKMKYTNMVIKETLRYRPPVLMVPYVVKKNFPVSPNYTAPKGAMLIPTLYPALHDPEVYENPDEFIPERWVEGSKASEAKKNWLVFGCGPHVCLGQTYVMITFAALLGKFALYTDFHHTVTPLSEKIKVFATIFPKDDLLLTFKKRDPITGEVFE

SEQ ID NO: 2 - DNA, 1617 bp, Saccharomyces cerevisiae
atgagttctgtcgcagaaaatataatacaacatgccactcataattctacgctacaccaattggctaaagaccagccctctgtaggcgtcactactgccttcagtatcctggatacacttaagtctatgtcatatttgaaaatatttgctactttaatctgtattcttttggtttgggaccaagttgcatatcaaatcaagaaaggttccatcgcaggtccaaagtttaagttctggcccatcatcggtccatttttggaatccttagatccaaagtttgaagaatataaggctaagtgggcatccggtccactttcatgtgtttctattttccataaatttgttgttatcgcatctactagagacttggcaagaaagatcttgcaatcttccaaattcgtcaaaccttgcgttgtcgatgttgctgtgaagatcttaagaccttgcaattgggtttttttggacggtaaagctcatactgattacagaaaatcattaaacggtcttttcactaaacaagctttggctcaatacttaccttcattggaacaaatcatggataagtacatggataagtttgttcgtttatctaaggagaataactacgagccccaggtctttttccatgaaatgagagaaattctttgcgccttatcattgaactctttctgtggtaactatattaccgaagatcaagtcagaaagattgctgatgattactatttggttacagcagcattggaattagtcaacttcccaattattatcccttacactaaaacatggtatggtaagaaaactgcagacatggccatgaagattttcgaaaactgtgctcaaatggctaaggatcatattgctgcaggtggtaagccagtttgtgttatggatgcttggtgtaagttgatgcacgatgcaaagaatagtaacgatgatgattctagaatctaccacagagagtttactaacaaggaaatctccgaagctgttttcactttcttatttgcttctcaagatgcctcttcttctttagcttgttggttgttccaaattgttgctgaccgtccagatgtcttagctaagatcagagaagaacaattggctgttcgtaacaatgacatgtctaccgaattgaacttggatttgattgagaaaatgaagtacaccaatatggtcataaaagaaactttgcgttacagacctcctgtcttaatggttccatatgttgttaagaagaatttcccagtttcccctaactataccgcaccaaaaggcgctatgttaattccaaccttatacccagctttacatgatcctgaagtttacgaaaatcccgatgagttcatccctgaaagatgggtagaaggctctaaggctagtgaagcaaagaagaattggttggtttttggttgtggtccacacgtttgcttaggtcaaacatatgtcatgattaccttcgccgctttgttgggtaaatttgcactatatactgatttccatcatacagtgactccattaagtgaaaaaatcaaggttttcgctacaattttcccaaaagatgatttgttactgactttcaaaaagagagacccaattactggagaagtcttcgaataa

SEQ ID NO: 3 - protein, 332 aa, Pelodiscus sinensis japonicus
MSVKELLIQNVHKEEHSHAHNKITVVGVGAVGMACAISILMKDLADELALVDVIEDKLRGEMLDLQHGSLFLRTPKIVSGKDYSVTAHSKLVIITAGARQQEGESRLNLVQRNVNIFKFIIPNVVKYSPDCMLLVVSNPVDILTYVAWKISGFPKHRVIGSGCNLDSARFRYLMGEKLGIHSLSCHGWIIGEHGDSSVPVWSGVNVAGVSLKALYPDLGTDADKEHWKEVHKQVVDSAYEVIKLKGYTSWAIGLSVADLAETVMKNLRRVHPISTMVKGMYGVSSDVFLSVPCVLGYAGITDVVKMTLKSEEEEKLRKSADTLWGIQKELQF

SEQ ID NO: 4 - protein, 332 aa, Ornithorhynchus anatinus
MAGVKEQLIQNLLKEEYAPQNKITVVGVGAVGMACAISILMKDLADELALVDVIEDKLKGEMMDLQHGSLFLRTPKIVSGKDYSVTANSKLVIITAGARQQEGESRLNLVQRNVNIFKFIIPNVVKYSPNCKLLVVSNPVDILTYVAWKISGFPKNRVIGSGCNLDSARFRYLMGERLGIHSTSCHGWVIGEHGDSSVPVWSGVNVAGVSLKNLHPDLGTDADKEQWKDVHKQVVDSAYEVIKLKGYTSWAIGLSVADLAESIVKNLRRVHPISTMIKGLYGIKDEVFLSVPCVLGQNGISDVVKITLKSEEEAHLKKSADTLWGIQKELQF

SEQ ID NO: 5 - protein, 332 aa, Tursiops truncatus
MATVKDQLIQNLLKEEHVPQNKITVVGVGAVGMACAISILMKDLADELALVDVIEDKLKGEMMDLQHGSLFLRTPKIVSGKDYSVTANSKLVIITAGARQQEGESRLNLVQRNVNIFKFIVPNIVKYSPHCKLLVVSNPVDILTYVAWKISGFPKNRVIGSGCNLDSARFRYLMGERLGVHPLSCHGWILGEHGDSSVPVWSGVNVAGVSLKNLHPELGTDADKEHWKAIHKQVVDSAYEVIKLKGYTSWAVGLSVADLAESIMKNLRRVHPISTMIKGLYGIKEDVFLSVPCILGQNGISDVVKVTLTPEEQACLKKSADTLWGIQKELQF

SEQ ID NO: 6 - protein, 332 aa, Rattus norvegicus
MAALKDQLIVNLLKEEQVPQNKITVVGVGAVGMACAISILMKDLADELALVDVIEDKLKGEMMDLQHGSLFLKTPKIVSSKDYSVTANSKLVIITAGARQQEGESRLNLVQRNVNIFKFIIPNVVKYSPQCKLLIVSNPVDILTYVAWKISGFPKNRVIGSGCNLDSARFRYLMGERLGVHPLSCHGWVLGEHGDSSVPVWSGVNVAGVSLKSLNPQLGTDADKEQWKDVHKQVVDSAYEVIKLKGYTSWAIGLSVADLAESIMKNLRRVHPISTMIKGLYGIKEDVFLSVPCILGQNGISDVVKVTLTPDEEARLKKSADTLWGIQKELQF

SEQ ID NO: 7 - protein, 332 aa, Bos Taurus
MATLKDQLIQNLLKEEHVPQNKITIVGVGAVGMACAISILMKDLADEVALVDVMEDKLKGEMMDLQHGSLFLRTPKIVSGKDYNVTANSRLVIITAGARQQEGESRLNLVQRNVNIFKFIIPNIVKYSPNCKLLVVSNPVDILTYVAWKISGFPKNRVIGSGCNLDSARFRYLMGERLGVHPLSCHGWILGEHGDSSVPVWSGVNVAGVSLKNLHPELGTDADKEQWKAVHKQVVDSAYEVIKLKGYTSWAIGLSVADLAESIMKNLRRVHPISTMIKGLYGIKEDVFLSVPCILGQNGISDVVKVTLTHEEEACLKKSADTLWGIQKELQF

SEQ ID NO: 8 - DNA, 999 bp, Pelodiscus sinensis japonicus
atgtccgtaaaggaactacttatacaaaacgtccataaggaggagcattctcacgctcacaataagataacagttgtaggagtaggtgcagtaggtatggcatgtgctatttcgatattaatgaaagacttggctgatgaactagccttggttgatgtgattgaggataagttacgtggagaaatgttagatttgcaacatggttcattgttcttgagaacccccaaaattgtctcgggtaaggattattcagtcactgctcattctaaactggttatcattacagcaggtgcaagacagcaagaaggggagagcagactaaatctggttcaacgtaatgtcaacatcttcaagtttatcatcccgaacgtagtaaaatacagtccagactgcatgttgcttgttgtgagtaatccagttgacatcttaacctatgttgcgtggaaaatcagtgggtttccaaaacatagggtgattggctcaggatgcaaccttgatagcgccaggtttaggtatctaatgggagaaaaattaggtattcactccttatcttgtcatggctggataataggcgaacatggtgattcttcggtacctgtttggtccggggttaatgtggctggtgttagtttaaaagcattatatcctgacctgggtactgatgccgataaagaacattggaaagaagtgcacaaacaagtggttgattctgcttacgaagttattaaacttaagggctacacttcttgggctataggtctatcagtagctgatttggcagaaaccgttatgaaaaatttaagaagagtccacccaatttccacgatggtcaagggtatgtacggtgttagctctgacgtcttcttatctgttccttgtgttttgggatatgcgggaattacagacgtcgtgaagatgacattgaaatcagaggaagaggaaaaactaagaaagtcagccgatactctgtggggcattcaaaaggaattgcagttttaa

SEQ ID NO: 9 - DNA, 999 bp, Bos Taurus
atggcaacattaaaagatcaactaatccagaatttgttgaaagaggagcatgttccacaaaacaaaatcacaatcgtcggcgtaggtgcagtaggtatggcttgtgccatatccatcttgatgaaagacttagctgatgaggtcgcgctggttgatgtaatggaggacaaacttaaaggagaaatgatggatcttcaacatggttcactctttttgagaactcctaaaattgtatccgggaaagattataacgttaccgccaattctagacttgttataatcacggctggtgcaagacaacaggaaggcgaatcaagacttaacttagttcagagaaacgtaaacattttcaagtttatcatcccaaatattgtaaaatactccccaaattgcaagttgctggttgtttcaaatcctgttgacatattgacttacgttgcttggaagatttcaggtttcccaaagaatagagtaatcggatctggttgcaatctcgattctgctcgttttaggtatctgatgggtgaaagattaggggttcatccattgagttgtcacggatggattctaggtgaacatggagatagttctgtgcctgtttggtcaggtgtcaacgtagcaggtgtctctttgaaaaatctacacccagaactaggaacagatgccgacaaggaacaatggaaggccgtccacaaacaagtggtggattctgcctacgaagtcatcaaattgaagggctacacatcttgggcaattggcttatccgtcgctgatctggctgaatcaataatgaaaaacctccgtagagtgcatcctataagtactatgattaagggtttatacgggatcaaggaagatgtttttctatctgtgccatgtattttgggccaaaatggaatttctgacgttgttaaagtgacacttactcatgaagaggaagcgtgtttgaaaaagagcgcagacaccttatggggcatccaaaaggaattacaattctaa

SEQ ID NO: 10 - protein, 563 aa, Saccharomyces cerevisiae
MSEITLGKYLFERLKQVNVNTVFGLPGDFNLSLLDKIYEVEGMRWAGNANELNAAYAADGYARIKGMSCIITTFGVGELSALNGIAGSYAEHVGVLHVVGVPSISAQAKQLLLHHTLGNGDFTVFHRMSANISETTAMITDIATAPAEIDRCIRTTYVTQRPVYLGLPANLVDLNVPAKLLQTPIDMSLKPNDAESEKEVIDTILALVKDAKNPVILADACCSRHDVKAETKKLIDLTQFPAFVTPMGKGSIDEQHPRYGGVYVGTLSKPEVKEAVESADLILSVGALLSDFNTGSFSYSYKTKNIVEFHSDHMKIRNATFPGVQMKFVLQKLLTTIADAAKGYKPVAVPARTPANAAVPASTPLKQEWMWNQLGNFLQEGDVVIAETGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEIMLPVFDAPQNLVEQAKLTAATNAKQ

SEQ ID NO: 11 - DNA, 1692 bp, Saccharomyces cerevisiae
atgtctgaaattactttgggtaaatatttgttcgaaagattaaagcaagtcaacgttaacaccgttttcggtttgccaggtgacttcaacttgtccttgttggacaagatctacgaagttgaaggtatgagatgggctggtaacgccaacgaattgaacgctgcttacgccgctgatggttacgctcgtatcaagggtatgtcttgtatcatcaccaccttcggtgtcggtgaattgtctgctttgaacggtattgccggttcttacgctgaacacgtcggtgttttgcacgttgttggtgtcccatccatctctgctcaagctaagcaattgttgttgcaccacaccttgggtaacggtgacttcactgttttccacagaatgtctgccaacatttctgaaaccactgctatgatcactgacattgctaccgccccagctgaaattgacagatgtatcagaaccacttacgtcacccaaagaccagtctacttaggtttgccagctaacttggtcgacttgaacgtcccagctaagttgttgcaaactccaattgacatgtctttgaagccaaacgatgctgaatccgaaaaggaagtcattgacaccatcttggctttggtcaaggatgctaagaacccagttatcttggctgatgcttgttgttccagacacgacgtcaaggctgaaactaagaagttgattgacttgactcaattcccagctttcgtcaccccaatgggtaagggttccattgacgaacaacacccaagatacggtggtgtttacgtcggtaccttgtccaagccagaagttaaggaagccgttgaatctgctgacttgattttgtctgtcggtgctttgttgtctgatttcaacaccggttctttctcttactcttacaagaccaagaacattgtcgaattccactccgaccacatgaagatcagaaacgccactttcccaggtgtccaaatgaaattcgttttgcaaaagttgttgaccactattgctgacgccgctaagggttacaagccagttgctgtcccagctagaactccagctaacgctgctgtcccagcttctaccccattgaagcaagaatggatgtggaaccaattgggtaacttcttgcaagaaggtgatgttgtcattgctgaaaccggtacctccgctttcggtatcaaccaaaccactttcccaaacaacacctacggtatctctcaagtcttatggggttccattggtttcaccactggtgctaccttgggtgctgctttcgctgctgaagaaattgatccaaagaagagagttatcttattcattggtgacggttctttgcaattgactgttcaagaaatctccaccatgatcagatggggcttgaagccatacttgttcgtcttgaacaacgatggttacaccattgaaaagttgattcacggtccaaaggctcaatacaacgaaattcaaggttgggaccacctatccttgttgccaactttcggtgctaaggactatgaaacccacagagtcgctaccaccggtgaatgggacaagttgacccaagacaagtctttcaacgacaactctaagatcagaatgattgaaatcatgttgccagtcttcgatgctccacaaaacttggttgaacaagctaagttgactgctgctaccaacgctaagcaataa

SEQ ID NO: 12 - protein, 591 aa, Saccharomyces cerevisiae
MLKYKPLLKISKNCEAAILRASKTRLNTIRAYGSTVPKSKSFEQDSRKRTQSWTALRVGAILAATSSVAYLNWHNGQIDNEPKLDMNKQKISPAEVAKHNKPDDCWVVINGYVYDLTRFLPNHPGGQDVIKFNAGKDVTAIFEPLHAPNVIDKYIAPEKKLGPLQGSMPPELVCPPYAPGETKEDIARKEQLKSLLPPLDNIINLYDFEYLASQTLTKQAWAYYSSGANDEVTHRENHNAYHRIFFKPKILVDVRKVDISTDMLGSHVDVPFYVSATALCKLGNPLEGEKDVARGCGQGVTKVPQMISTLASCSPEEIIEAAPSDKQIQWYQLYVNSDRKITDDLVKNVEKLGVKALFVTVDAPSLGQREKDMKLKFSNTKAGPKAMKKTNVEESQGASRALSKFIDPSLTWKDIEELKKKTKLPIVIKGVQRTEDVIKAAEIGVSGVVLSNHGGRQLDFSRAPIEVLAETMPILEQRNLKDKLEVFVDGGVRRGTDVLKALCLGAKGVGLGRPFLYANSCYGRNGVEKAIEILRDEIEMSMRLLGVTSIAELKPDLLDLSTLKARTVGVPNDVLYNEVYEGPTLTEFEDA

SEQ ID NO: 13 - DNA, 1776 bp, Saccharomyces cerevisiae
atgctaaaatacaaacctttactaaaaatctcgaagaactgtgaggctgctatcctcagagcgtctaagactagattgaacacaatccgcgcgtacggttctaccgttccaaaatccaagtcgttcgaacaagactcaagaaaacgcacacagtcatggactgccttgagagtcggtgcaattctagccgctactagttccgtggcgtatctaaactggcataatggccaaatagacaacgagccgaaactggatatgaataaacaaaagatttcgcccgctgaagttgccaagcataacaagcccgatgattgttgggttgtgatcaatggttacgtatacgacttaacgcgattcctaccaaatcatccaggtgggcaggatgttatcaagtttaacgccgggaaagatgtcactgctatttttgaaccactacatgctcctaatgtcatcgataagtatatagctcccgagaaaaaattgggtccccttcaaggatccatgcctcctgaacttgtctgtcctccttatgctcctggtgaaactaaggaagatatcgctagaaaagaacaactaaaatcgctgctacctcctctagataatattattaacctttacgactttgaatacttggcctctcaaactttgactaaacaagcgtgggcctactattcctccggtgctaacgacgaagttactcacagagaaaaccataatgcttatcataggatttttttcaaaccaaagatccttgtagatgtacgcaaagtagacatttcaactgacatgttgggttctcatgtggatgttcccttctacgtgtctgctacagctttgtgtaaactgggaaaccccttagaaggtgaaaaagatgtcgccagaggttgtggccaaggtgtgacaaaagtcccacaaatgatatctactttggcttcatgttcccctgaggaaattattgaagcagcaccctctgataaacaaattcaatggtaccaactatatgttaactctgatagaaagatcactgatgatttggttaaaaatgtagaaaagctgggtgtaaaggcattatttgtcactgtggatgctccaagtttaggtcaaagagaaaaagatatgaagctgaaattttccaatacaaaggctggtccaaaagcgatgaagaaaactaatgtagaagaatctcaaggtgcttcgagagcgttatcaaagtttattgacccctctttgacttggaaagatatagaagagttgaagaaaaagacaaaactacctattgttatcaaaggtgttcaacgtaccgaagatgttatcaaagcagcagaaatcggtgtaagtggggtggttctatccaatcatggtggtagacaattagatttttcaagggctcccattgaagtcctggctgaaaccatgccaatcctggaacaacgtaacttgaaggataagttggaagttttcgtggacggtggtgttcgtcgtggtacagatgtcttgaaagcgttatgtctaggtgctaaaggtgttggtttgggtagaccattcttgtatgcgaactcatgctatggtcgtaatggtgttgaaaaagccattgaaattttaagagatgaaattgaaatgtctatgagactattaggtgttactagcattgcggaattgaagcctgatcttttagatctatcaacactaaaggcaagaacagttggagtaccaaacgacgtgctgtataatgaagtttatgagggacctactttaacagaatttgaggatgcatga

SEQ ID NO: 14 - protein, 391 aa, Saccharomyces cerevisiae
MSAAADRLNLTSGHLNAGRKRSSSSVSLKAAEKPFKVTVIGSGNWGTTIAKVVAENCKGYPEVFAPIVQMWVFEEEINGEKLTEIINTRHQNVKYLPGITLPDNLVANPDLIDSVKDVDIIVFNIPHQFLPRICSQLKGHVDSHVRAISCLKGFEVGAKGVQLLSSYITEELGIQCGALSGANIATEVAQEHWSETTVAYHIPKDFRGEGKDVDHKVLKALFHRPYFHVSVIEDVAGISICGALKNVVALGCGFVEGLGWGNNASAAIQRVGLGEIIRFGQMFFPESREETYYQESAGVADLITTCAGGRNVKVARLMATSGKDAWECEKELLNGQSAQGLITCKEVHEWLETCGSVEDFPLFEAVYQIVYNNYPMKNLPDMIEELDLHED

SEQ ID NO: 15 - DNA, 1176 bp, Saccharomyces cerevisiae
atgtctgctgctgctgatagattaaacttaacttccggccacttgaatgctggtagaaagagaagttcctcttctgtttctttgaaggctgccgaaaagcctttcaaggttactgtgattggatctggtaactggggtactactattgccaaggtggttgccgaaaattgtaagggatacccagaagttttcgctccaatagtacaaatgtgggtgttcgaagaagagatcaatggtgaaaaattgactgaaatcataaatactagacatcaaaacgtgaaatacttgcctggcatcactctacccgacaatttggttgctaatccagacttgattgattcagtcaaggatgtcgacatcatcgttttcaacattccacatcaatttttgccccgtatctgtagccaattgaaaggtcatgttgattcacacgtcagagctatctcctgtctaaagggttttgaagttggtgctaaaggtgtccaattgctatcctcttacatcactgaggaactaggtattcaatgtggtgctctatctggtgctaacattgccaccgaagtcgctcaagaacactggtctgaaacaacagttgcttaccacattccaaaggatttcagaggcgagggcaaggacgtcgaccataaggttctaaaggccttgttccacagaccttacttccacgttagtgtcatcgaagatgttgctggtatctccatctgtggtgctttgaagaacgttgttgccttaggttgtggtttcgtcgaaggtctaggctggggtaacaacgcttctgctgccatccaaagagtcggtttgggtgagatcatcagattcggtcaaatgtttttcccagaatctagagaagaaacatactaccaagagtctgctggtgttgctgatttgatcaccacctgcgctggtggtagaaacgtcaaggttgctaggctaatggctacttctggtaaggacgcctgggaatgtgaaaaggagttgttgaatggccaatccgctcaaggtttaattacctgcaaagaagttcacgaatggttggaaacatgtggctctgtcgaagacttcccattatttgaagccgtataccaaatcgtttacaacaactacccaatgaagaacctgccggacatgattgaagaattagatctacatgaagattag

SEQ ID NO: 16 - protein, 348 aa, Saccharomyces cerevisiae
MSIPETQKGVIFYESHGKLEYKDIPVPKPKANELLINVKYSGVCHTDLHAWHGDWPLPVKLPLVGGHEGAGVVVGMGENVKGWKIGDYAGIKWLNGSCMACEYCELGNESNCPHADLSGYTHDGSFQQYATADAVQAAHIPQGTDLAQVAPILCAGITVYKALKSANLMAGHWVAISGAAGGLGSLAVQYAKAMGYRVLGIDGGEGKEELFRSIGGEVFIDFTKEKDIVGAVLKATDGGAHGVINVSVSEAAIEASTRYVRANGTTVLVGMPAGAKCCSDVFNQVVKSISIVGSYVGNRADTREALDFFARGLVKSPIKVVGLSTLPEIYEKMEKGQIVGRYVVDTSK

SEQ ID NO: 17 - DNA, 1047 bp, Saccharomyces cerevisiae
atgtctatcccagaaactcaaaaaggtgttatcttctacgaatcccacggtaagttggaatacaaagatattccagttccaaagccaaaggccaacgaattgttgatcaacgttaaatactctggtgtctgtcacactgacttgcacgcttggcacggtgactggccattgccagttaagctaccattagtcggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaacaatacgctaccgctgacgctgttcaagccgctcacattcctcaaggtaccgacttggcccaagtcgcccccatcttgtgtgctggtatcaccgtctacaaggctttgaagtctgctaacttgatggccggtcactgggttgctatctccggtgctgctggtggtctaggttctttggctgttcaatacgccaaggctatgggttacagagtcttgggtattgacggtggtgaaggtaaggaagaattattcagatccatcggtggtgaagtcttcattgacttcactaaggaaaaggacattgtcggtgctgttctaaaggccactgacggtggtgctcacggtgtcatcaacgtttccgtttccgaagccgctattgaagcttctaccagatacgttagagctaacggtaccaccgttttggtcggtatgccagctggtgccaagtgttgttctgatgtcttcaaccaagtcgtcaagtccatctctattgttggttcttacgtcggtaacagagctgacaccagagaagctttggacttcttcgccagaggtttggtcaagtctccaatcaaggttgtcggcttgtctaccttgccagaaatttacgaaaagatggaaaagggtcaaatcgttggtagatacgttgttgacacttctaaataa

SEQ ID NO: 18 - protein, 500 aa, Saccharomyces cerevisiae
MTKLHFDTAEPVKITLPNGLTYEQPTGLFINNKFMKAQDGKTYPVEDPSTENTVCEVSSATTEDVEYAIECADRAFHDTEWATQDPRERGRLLSKLADELESQIDLVSSIEALDNGKTLALARGDVTIAINCLRDAAAYADKVNGRTINTGDGYMNFTTLEPIGVCGQIIPWNFPIMMLAWKIAPALAMGNVCILKPAAVTPLNALYFASLCKKVGIPAGVVNIVPGPGRTVGAALTNDPRIRKLAFTGSTEVGKSVAVDSSESNLKKITLELGGKSAHLVFDDANIKKTLPNLVNGIFKNAGQICSSGSRIYVQEGIYDELLAAFKAYLETEIKVGNPFDKANFQGAITNRQQFDTIMNYIDIGKKEGAKILTGGEKVGDKGYFIRPTVFYDVNEDMRIVKEEIFGPVVTVAKFKTLEEGVEMANSSEFGLGSGIETESLSTGLKVAKMLKAGTVWINTYNDFDSRVPFGGVKQSGYGREMGEEVYHAYTEVKAVRIKL

SEQ ID NO: 19 - DNA, 1503 bp, Saccharomyces cerevisiae
atgactaagctacactttgacactgctgaaccagtcaagatcacacttccaaatggtttgacatacgagcaaccaaccggtctattcattaacaacaagtttatgaaagctcaagacggtaagacctatcccgtcgaagatccttccactgaaaacaccgtttgtgaggtctcttctgccaccactgaagatgttgaatatgctatcgaatgtgccgaccgtgctttccacgacactgaatgggctacccaagacccaagagaaagaggccgtctactaagtaagttggctgacgaattggaaagccaaattgacttggtttcttccattgaagctttggacaatggtaaaactttggccttagcccgtggggatgttaccattgcaatcaactgtctaagagatgctgctgcctatgccgacaaagtcaacggtagaacaatcaacaccggtgacggctacatgaacttcaccaccttagagccaatcggtgtctgtggtcaaattattccatggaactttccaataatgatgttggcttggaagatcgccccagcattggccatgggtaacgtctgtatcttgaaacccgctgctgtcacacctttaaatgccctatactttgcttctttatgtaagaaggttggtattccagctggtgtcgtcaacatcgttccaggtcctggtagaactgttggtgctgctttgaccaacgacccaagaatcagaaagctggcttttaccggttctacagaagtcggtaagagtgttgctgtcgactcttctgaatctaacttgaagaaaatcactttggaactaggtggtaagtccgcccatttggtctttgacgatgctaacattaagaagactttaccaaatctagtaaacggtattttcaagaacgctggtcaaatttgttcctctggttctagaatttacgttcaagaaggtatttacgacgaactattggctgctttcaaggcttacttggaaaccgaaatcaaagttggtaatccatttgacaaggctaacttccaaggtgctatcactaaccgtcaacaattcgacacaattatgaactacatcgatatcggtaagaaagaaggcgccaagatcttaactggtggcgaaaaagttggtgacaagggttacttcatcagaccaaccgttttctacgatgttaatgaagacatgagaattgttaaggaagaaatttttggaccagttgtcactgtcgcaaagttcaagactttagaagaaggtgtcgaaatggctaacagctctgaattcggtctaggttctggtatcgaaacagaatctttgagcacaggtttgaaggtggccaagatgttgaaggccggtaccgtctggatcaacacatacaacgattttgactccagagttccattcggtggtgttaagcaatctggttacggtagagaaatgggtgaagaagtctaccatgcatacactgaagtaaaagctgtcagaattaagttgtaa

SEQ ID NO: 20 - protein, 316 aa, Escherichia coli
MSKRKVAIIGSGNIGTDLMIKILRHGQHLEMAVMVGIDPQSDGLARARRMGVATTHEGVIGLMNMPEFADIDIVFDATSAGAHVKNDAALREAKPDIRLIDLTPAAIGPYCVPVVNLEANVDQLNVNMVTCGGQATIPMVAAVSRVARVHYAEIIASIASKSAGPGTRANIDEFTETTSRAIEVVGGAAKGKAIIVLNPAEPPLMMRDTVYVLSDEASQDDIEASINEMAEAVQAYVPGYRLKQRVQFEVIPQDKPVNLPGVGQFSGLKTAVWLEVEGAAHYLPAYAGNLDIMTSSALATAEKMAQSLARKAGEAA

SEQ ID NO: 21 - DNA, 951 bp, Escherichia coli
atgagtaagcgtaaagtcgccattatcggttctggcaacattggtaccgatctgatgattaaaattttgcgtcacggtcagcatctggagatggcggtgatggttggcattgatcctcagtccgacggtctggcgcgcgccagacgtatgggcgtcgccaccacccatgaaggggtgatcggactgatgaacatgcctgaatttgctgatatcgacattgtatttgatgcgaccagcgccggtgctcatgtgaaaaacgatgccgctttacgcgaagcgaaaccggatattcgcttaattgacctgacgcctgctgccatcggcccttactgcgtgccggtggttaacctcgaggcgaacgtcgatcaactgaacgtcaacatggtcacctgcggcggccaggccaccattccaatggtggcggcagtttcacgcgtggcgcgtgttcattacgccgaaattatcgcttctatcgccagtaaatctgccggacctggcacgcgtgccaatatcgatgaatttacggaaaccacttcccgagccattgaagtggtgggcggcgcggcaaaagggaaggcgattattgtgcttaacccagcagagccaccgttgatgatgcgtgacacggtgtatgtattgagcgacgaagcttcacaagatgatatcgaagcctcaatcaatgaaatggctgaggcggtgcaggcttacgtaccgggttatcgcctgaaacagcgcgtgcagtttgaagttatcccgcaggataaaccggtcaatttaccgggcgtggggcaattctccggactgaaaacagcggtctggctggaagtcgaaggcgcagcgcattatctgcctgcctatgcgggcaacctcgacattatgacttccagtgcgctggcgacagcggaaaaaatggcccagtcactggcgcgcaaggcaggagaagcggcatga

SEQ ID NO: 22 - DNA, 954 bp, Artificial Sequence
  Synthetic S. cevisiae optimized MhpF
atgtcaaagcgaaaagtagctatcataggttcaggtaatattggtactgatttgatgatcaaaatcctgagacatggccagcacttggagatggccgtcatggttggtatcgacccacaatccgatggcttagctagagctaggagaatgggtgttgccacaactcacgaaggggttattggcttaatgaacatgccagaatttgcagacatcgatatagtttttgatgctactagtgcaggggcacatgtgaaaaacgacgcggctttaagagaagccaagccagatattagattaattgatcttacccctgctgctataggtccttactgcgttcctgtagttaaccttgaagctaatgtggaccagttgaacgtgaatatggttacatgtggtggccaagctaccataccaatggttgctgctgtctctagagtggccagagtacattatgccgagatcattgcgtctatcgcatctaagtctgccggtcctggaacaagggctaacatcgatgagttcactgagacaacctctagagctatcgaagtagtaggaggcgcagcaaaaggtaaagcgatcattgttttgaatcctgccgaaccacctttgatgatgagagatacggtctacgtgctatcagatgaagcttcccaggatgacattgaagctagcattaatgagatggcagaagccgttcaagcatacgtgccaggatatagactcaaacaaagagtccaatttgaggtcattccacaagacaagccagttaatctcccaggggtcggtcaattctcaggactaaaaactgctgtttggttagaagtagaaggagctgctcattacctaccagcctacgccggtaatttggatataatgacatcttccgctcttgcaacagcagaaaagatggcacaaagtctggcccgtaaggcaggagaagcggcataataa

SEQ ID NO: 23 - DNA, 289 bp, Artificial Sequence
  Synthetic CYC promoter
atttggcgagcgttggttggtggatcaagcccacgcgtaggcaatcctcgagcagatccgccaggcgtgtatatatagcgtggatggccaggcaactttagtgctgacacatacaggcatatatatatgtgtgcgacgacacatgatcatatggcatgcatgtgctctgtatgtatataaaactcttgttttcttcttttctctaaatattctttccttatacattaggacctttgcagcataaattactatacttctatagacacgcaaacacaaatacacacactaa

SEQ ID NO: 24 - DNA, 401 bp, Artificial Sequence
  Synthetic TEF promoter
atagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaattttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttt

SEQ ID NO: 25 - DNA, 655 bp, Artificial Sequence
  Synthetic GPD promoter
agtttatcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcgacggat

SEQ ID NO: 26 - DNA, 1468 bp, Artificial Sequence
  Synthetic ADH promoter
gccgggatcgaagaaatgatggtaaatgaaataggaaatcaaggagcatgaaggcaaaagacaaatataagggtcgaacgaaaaataaagtgaaaagtgttgatatgatgtatttggctttgcggcgccgaaaaaacgagtttacgcaattgcacaatcatgctgactctgtggcggacccgcgctcttgccggcccggcgataacgctgggcgtgaggctgtgcccggcggagttttttgcgcctgcattttccaaggtttaccctgcgctaaggggcgagattggagaagcaataagaatgccggttggggttgcgatgatgacgaccacgacaactggtgtcattatttaagttgccgaaagaacctgagtgcatttgcaacatgagtatactagaagaatgagccaagacttgcgagacgcgagtttgccggtggtgcgaacaatagagcgaccatgaccttgaaggtgagacgcgcataaccgctagagtactttgaagaggaaacagcaatagggttgctaccagtataaatagacaggtacatacaacactggaaatggttgtctgtttgagtacgctttcaattcatttgggtgtgcactttattatgttacaatatggaagggaactttacacttctcctatgcacatatattaattaaagtccaatgctagtagagaaggggggtaacacccctccgcgctcttttccgatttttttctaaaccgtggaatatttcggatatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactccaagctggccgc

SEQ ID NO: 27 - DNA, 292 bp, Artificial Sequence
  Synthetic CCW12 promoter
ttcgcggccacctacgccgctatctttgcaacaactatctgcgataactcagcaaattttgcatattcgtgttgcagtattgcgataatgggagtcttacttccaacataacggcagaaagaaatgtgagaaaattttgcatcctttgcctccgttcaagtatataaagtcggcatgcttgataatctttctttccatcctacattgttctaattattcttattctcctttattctttcctaacataccaagaaattaatcttctgtcattcgcttaaacactatatcaata

SEQ ID NO: 28 - DNA, 252 bp, Artificial Sequence
  Synthetic CYC1 terminator
tcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggcc

SEQ ID NO: 29 - DNA, 247 bp, Artificial Sequence
  Synthetic TPS terminator
acccgatgcaaatgagacgatcgtctattcctggtccggttttctctgccctctcttctattcactttttttatactttatataaaattatataaatgacataactgaaacgccacacgtcctctcctattcgttaacgcctgtctgtagcgctgttactgaagctgcgcaagtagttttttcaccgtataggccctctttttctctctctttctttctctcccgcgctgatctcttcttcgaaaca

SEQ ID NO: 30 - DNA, 355 bp, Artificial Sequence
  Synthetic TPS terminator
acccgatgcaaatgagacgatcgtctattcctggtccggttttctctgccctctcttctattcactttttttatactttatataaaattatataaatgacataactgaaacgccacacgtcctctcctattcgttaacgcctgtctgtagcgctgttactgaagctgcgcaagtagttttttcaccgtataggccctctttttctctctctttctttctctcccgcgctgatctcttcttcgaaacatcatgaataaaaagaaaaaggaaatcaagaaaaaaaagccataatttatcccacatttttttttattgtcgctgttcacaccgcataacgaagatattggctagctaa

SEQ ID NO: 31 - DNA, 31 bp, Artificial Sequence
  Synthetic primer
cgagctcttcgcggccacctacgccgctatc

SEQ ID NO: 32 - DNA, 32 bp, Artificial Sequence
  Synthetic primer
gctctagatattgatatagtgtttaagcgaat

SEQ ID NO: 33 - DNA, 27 bp, Artificial Sequence
  Synthetic primer
cggccatggcgggagctcgcatgcaag

SEQ ID NO: 34 - DNA, 28 bp, Artificial Sequence
  Synthetic primer
cgggatatcactagtgagctcgctccgc

SEQ ID NO: 35 - DNA, 2321 bp, Artificial Sequence
  Synthetic HPH cassette
gccgggagagctcgcatgcaagtaacctattcaaagtaatatctcatacatgtttcatgagggtaacaacatgcgactgggtgagcatatgttccgctgatgtgatgtgcaagataaacaagcaaggcagaaactaacttcttcttcatgtaataaacacaccccgcgtttatttacctatctctaaacttcaacaccttatatcataactaatatttcttgagataagcacactgcacccataccttccttaaaaacgtagcttccagtttttggtggttccggcttccttcccgattccgcccgctaaacgcatatttttgttgcctggtggcatttgcaaaatgcataacctatgcatttaaaagattatgtatgctcttctgacttttcgtgtgatgaggctcgtggaaaaaatgaataatttatgaatttgagaacaattttgtgttgttacggtattttactatggaataatcaatcaattgaggattttatgcaaatatcgtttgaatatttttccgaccctttgagtacttttcttcataattgcataatattgtccgctgcccctttttctgttagacggtgtcttgatctacttgctatcgttcaacaccaccttattttctaactattttttttttagctcatttgaatcagcttatggtgatggcacatttttgcataaacctagctgtcctcgttgaacataggaaaaaaaaatatataaacaaggctctttcactctccttgcaatcagatttgggtttgttccctttattttcatatttcttgtcatattcctttctcaattattattttctactcataacctcacgcaaaataacacagtcaaatcctcgagatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaggatccgtagatacattgatgctatcaatcaagagaactggaaagattgtgtaaccttgaaaaacggtgaaacttacgggtccaagattgtctacagattttcctgatttgccagcttactatccttcttgaaaatatgcactctatatcttttagttcttaattgcaacacatagatttgctgtataacgaattttatgctattttttaaatttggagttcagtgataaaagtgtcacagcgaatttcctcacatgtagggaccgaattgtttacaagttctctgtaccaccatggagacatcaaaaattgaaaatctatggaaagatatggacggtagcaacaagaatatagcacgagccgcggagcgagctcggccgcactagtgatatcccgcggccatggcggccgggag

SEQ ID NO: 36 - DNA, 18 bp, Artificial Sequence
  Synthetic primer
gaaacagctatgaccatg

SEQ ID NO: 37 - DNA, 32 bp, Artificial Sequence
  Synthetic primer
gacatgacgagctcgaattgggtaccggccgc

SEQ ID NO: 38 - DNA, 4173 bp, Artificial Sequence
  Synthetic pUC57-Ura3HA vector
gatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatatcggatcccgacgagctgcaccgcggtggcggccgtatcttttacccatacgatgttcctgactatgcgggctatccctatgacgtcccggactatgcaggatcctatccatatgacgttccagattacgctgctcagtgcggccgcctgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtagcggccgcgaatttgagcttatcttttacccatacgatgttcctgactatgcgggctatccctatgacgtcccggactatgcaggatcctatccatatgacgttccagattacgctactagcggggggcccggtgacgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggt

SEQ ID NO: 39 - DNA, 62 bp, Artificial Sequence
  Synthetic primer
gcttataaaactttaactaataattagagattaaatcgcttaaggtttcccgactggaaagc

SEQ ID NO: 40 - DNA, 64 bp, Artificial Sequence
  Synthetic primer
ctactcataacctcacgcaaaataacacagtcaaatcaatcaaaccagtcacgacgttgtaaaa

SEQ ID NO: 41 - DNA, 20 bp, Artificial Sequence
  Synthetic primer
ggacgtaaagggtagcctcc

SEQ ID NO: 42 - DNA, 22 bp, Artificial Sequence
  Synthetic primer
gaagcggacccagacttaagcc

SEQ ID NO: 43 - DNA, 65 bp, Artificial Sequence
  Synthetic primer
ccgaaatgattccctttcctgcacaacacgagatctttcacgcatccagtcacgacgttgtaaaa

SEQ ID NO: 44 - DNA, 64 bp, Artificial Sequence
  Synthetic primer
aaagtagccttaaagctaggctataatcatgcatcctcaaattctaggtttcccgacggaaagc

SEQ ID NO: 45 - DNA, 25 bp, Artificial Sequence
  Synthetic primer
cgcaagaacgtagtatccacatgcc

SEQ ID NO: 46 - DNA, 21 bp, Artificial Sequence
  Synthetic primer
ggatatttacagaacgatgcg

SEQ ID NO: 47 - DNA, 70 bp, Artificial Sequence
  Synthetic primer
ccctatgtctctggccgatcacgcgccattgtccctcagaaacaaatcaaccagtcacgacgttgtaaaa

SEQ ID NO: 48 - DNA, 70 bp, Artificial Sequence
  Synthetic primer
tagaagcaactgtgccgacagcctctgaatgagtggtgttgtaaccacccaggtttcccgactggaaagc

SEQ ID NO: 49 - DNA, 25 bp, Artificial Sequence
  Synthetic primer
tcaatgagactgttgtcctcctact

SEQ ID NO: 50 - DNA, 24 bp, Artificial Sequence
  Synthetic primer
tacatccttgtcgagccttgggca

SEQ ID NO: 51 - DNA, 75 bp, Artificial Sequence
  Synthetic primer
acaatatttcaagctataccaagcatacaatcaactatctcatatacaatgggccgcaaattaaagccttcgagc

SEQ ID NO: 52 - DNA, 75 bp, Artificial Sequence
  Synthetic primer
aatcataagaaattcgcttatttagaagtgtcaacaacgtatctaccaacgactaaagggaacaaaagctggagc

SEQ ID NO: 53 - DNA, 18 bp, Artificial Sequence
  Synthetic primer
tgctgtcttgctatcaag

SEQ ID NO: 54 - DNA, 19 bp, Artificial Sequence
  Synthetic primer
caggaaagagttactcaag

SEQ ID NO: 55 - DNA, 5779 bp, Artificial Sequence
  Synthetic pUC19-His-MhpF
tcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcagtttatcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcgacggattctagaactagtggatccatgtcaaagcgaaaagtagctatcataggttcaggtaatattggtactgatttgatgatcaaaatcctgagacatggccagcacttggagatggccgtcatggttggtatcgacccacaatccgatggcttagctagagctaggagaatgggtgttgccacaactcacgaaggggttattggcttaatgaacatgccagaatttgcagacatcgatatagtttttgatgctactagtgcaggggcacatgtgaaaaacgacgcggctttaagagaagccaagccagatattagattaattgatcttacccctgctgctataggtccttactgcgttcctgtagttaaccttgaagctaatgtggaccagttgaacgtgaatatggttacatgtggtggccaagctaccataccaatggttgctgctgtctctagagtggccagagtacattatgccgagatcattgcgtctatcgcatctaagtctgccggtcctggaacaagggctaacatcgatgagttcactgagacaacctctagagctatcgaagtagtaggaggcgcagcaaaaggtaaagcgatcattgttttgaatcctgccgaaccacctttgatgatgagagatacggtctacgtgctatcagatgaagcttcccaggatgacattgaagctagcattaatgagatggcagaagccgttcaagcatacgtgccaggatatagactcaaacaaagagtccaatttgaggtcattccacaagacaagccagttaatctcccaggggtcggtcaattctcaggactaaaaactgctgtttggttagaagtagaaggagctgctcattacctaccagcctacgccggtaatttggatataatgacatcttccgctcttgcaacagcagaaaagatggcacaaagtctggcccgtaaggcaggagaagcggcataataaatcctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccaattcgagctcggtacccggggatcctctagagtcgacaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggg

SEQ ID NO: 56 - DNA, 31 bp, Artificial Sequence
  Synthetic primer
cctcctgagtcgacaattcccgttttaagag

SEQ ID NO: 57 - DNA, 30 bp, Artificial Sequence
  Synthetic primer
cgaccgtggtcgacccgtcgagttcaagag

SEQ ID NO: 58 - DNA, 64 bp, Artificial Sequence
  Synthetic primer
caagaaacatctttaacatacacaaacacatactatcagaatacccagtcacgacgttgtaaaa

SEQ ID NO: 59 - DNA, 65 bp, Artificial Sequence
  Synthetic primer
gtattttgtgtatatgacggaaagaaatgcaggttggtacattacaggtttcccgactggaaagc

SEQ ID NO: 60 - DNA, 26 bp, Artificial Sequence
  Synthetic primer
gacagtctagcaaacagtagtagtcc

SEQ ID NO: 61 - DNA, 19 bp, Artificial Sequence
  Synthetic primer
tgacgtaagaccaagtaag

SEQ ID NO: 62 - DNA, 59 bp, Artificial Sequence
  Synthetic primer
gtttcgacggattctagaaaacaatgagttctgtcgcagaaaatataatacaacatgcc

SEQ ID NO: 63 - DNA, 45 bp, Artificial Sequence
  Synthetic primer
taaggataagcagaaccgttattcgaagacttctccagtaattgg

SEQ ID NO: 64 - DNA, 59 bp, Artificial Sequence
  Synthetic primer
aatcttgtgctattgcagtcctcttttatatacagtataatacgactcactatagggcg

SEQ ID NO: 65 - DNA, 59 bp, Artificial Sequence
  Synthetic primer
atgcgaattgcgtaattcacggcgataacgtagtattaattaaccctcactaaagggaa

SEQ ID NO: 66 - DNA, 19 bp, Artificial Sequence
  Synthetic primer
gcccacaacttatcaagtg

SEQ ID NO: 67 - DNA, 19 bp, Artificial Sequence
  Synthetic primer
ttataagacaagcgcaggg